(19) United States
(12) Patent Application Publication
(10) Pub. No.: US 2012/0226119 A1
(43) Pub. Date: Sep. 6, 2012

(54) VIVO TUMOR VASCULATURE IMAGING

(75) Inventors: Michael Dobosz, Penzberg (DE); Christian Kein, Bonstetten (CH); Johannes Sam, Muenchen (DE); Werner Scheuer, Penzberg (DE)

(73) Assignee: Hoffmann-La Roche Inc., Nutley, NJ (US)

(21) Appl. No.: 13/383,197

(22) PCT Filed: Jul. 9, 2010
(86) PCT No.: PCT/EP2010/059858

§ 371 Date: May 22, 2012

(30) Foreign Application Priority Data
Jul. 9, 2009 (EP) 09165031.7

(51) Int. Cl. A61B 5/02; A61B 6/00

(52) U.S. Cl. 600/317

(57) ABSTRACT

The present invention relates to non-invasive methods of and uses for in vivo imaging tumor vasculature in a subject comprising detecting a fluorescence labelled anti-CD31 antibody. In a further aspect the present invention relates to non-invasive methods of and uses for in vivo monitoring the therapeutic efficacy of an anti-angiogenic agent in a subject comprising detecting a fluorescence labelled anti-CD31 antibody. In addition, a kit for use in the methods of the present invention is provided which comprises a fluorescence labelled anti-CD31 antibody and means for near-infrared fluorescence imaging to detect the antibody in a subject.

The present invention relates to non-invasive methods of and uses for in vivo imaging tumor vasculature in a subject comprising detecting a fluorescence labelled anti-CD31 antibody. In a further aspect the present invention relates to non-invasive methods of and uses for in vivo monitoring the therapeutic efficacy of an anti-angiogenic agent in a subject comprising detecting a fluorescence labelled anti-CD31 antibody. In addition, a kit for use in the methods of the present invention is provided which comprises a fluorescence labelled anti-CD31 antibody and means for near-infrared fluorescence imaging to detect the antibody in a subject.

Blood vessel formation occurs through the process of vasculogenesis and angiogenesis. Vasculogenesis is the differentiation de novo of vascular endothelial cells from precursor cells, known as angioblasts, during embryonic development.

This process differs from the process of angiogenesis, which is a remodelling process characterized by the sprouting of new blood vessels from pre-existing ones (Folkman and D'Amore, Cell 1996; Vol. 87: 1153-1155; Risau, Nature 1997; Vol. 386: 671-674). It has always been a widely held belief that vasculogenesis occurs only during early embryogenesis. Recent studies revealed the existence of endothelial progenitor stem cells and these bone marrow derived cells have been found to be incorporated into neovascular foci in the adult, including injured corneas, ischemic hind limbs and tumor vasculature, suggesting that vasculogenesis can occur in the adult (Asahara et al., Science 1997; Vol. 275: 964-967; Shi et al., Blood 1998; Vol. 92: 362-367).

Angiogenesis, however, occurs during embryogenesis and to a limited extent in the adult, for example in the female reproductive system, in physiological wound healing and in pathological disease processes such as cancer (Klagsbrun and Moses, Chemistry & Biology 1999; Vol. 6:217-224).

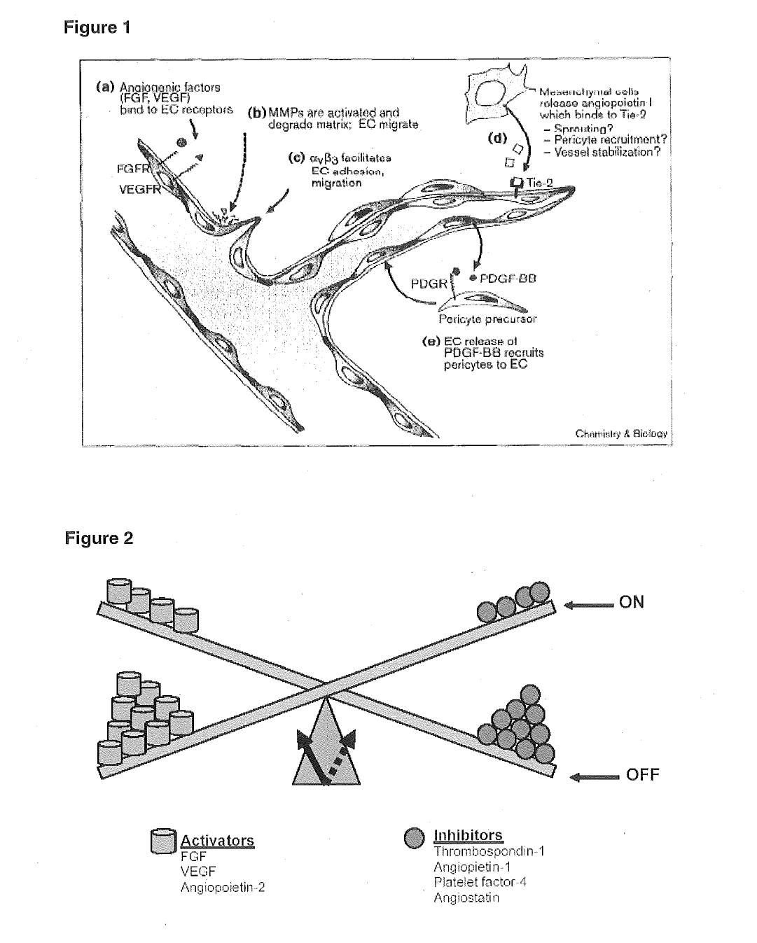

Angiogenesis is stimulated by various growth factors such as vascular endothelial growth factor (VEGF) and fibroblast growth factor (FGF). An early response to angiogenesis-stimulating factors is the degradation of the endothelial cell basement membrane by proteases such as members of the matrix metalloproteinase (MMP) family. MMPs degrade collagen and other extracellular matrix components (see FIG. 1). Destroying the basement membrane barrier, allows endothelial cells to migrate from pre-existing vessels towards angiogenic stimuli and to proliferate (Marsh, Stem Cells 1997; Vol. 15: 180-189). Vascular cell-adhesion molecules contribute to endothelial cell migration by mediating cell-extracellular-matrix interactions. One of the major mediators is the integrin αᵥβ₃, a receptor for proteins such as fibronectin. Antagonists of integrin αᵥβ₃ inhibit the growth of new blood vessels, suggesting that cell adhesion is a critical step in angiogenesis (Brooks et al., Cell 1994; Vol. 79: 1157-1164). Following migration and proliferation, endothelial cells assemble into tubes with a patent lumen. An important step in producing a mature blood vessel is the recruitment of mesenchymal cells and their subsequent differentiation into smooth muscle cell-like pericytes that are thought to stabilize the newly forming vasculature. These pericytes are gradually attracted by an endothelial plexus leading to progressive covering of the vascular tree (Benjamin et al., Development 1998; Vol. 125:1591-1598). During this period, regression of uncovered capillaries occurs, which ceases after acquisition of a pericyte coating. This later phase of blood vessel formation, therefore, is characterized by remodelling steps in which unprotected vessels are pruned and pericyte-covered vessels become stabilized and less prone to destabilization and regression. Platelet derived growth factor (PDGF) probably derived from an endothelial cell source, recruits pericytes to associate with endothelial cells to increase blood vessel stability (Lindahl et al., Science 1997; Vol. 277: 242-245). The above described processes in angiogenesis are also illustrated in FIG. 1.

Vasculogenesis and angiogenesis, in particular neo-angiogenesis are processes occurring not only in normal development, but also in abnormal development such as in tumor development. Namely, the proliferation and survival of tumor cells are dependent on an adequate supply of growth factors and the removal of toxic molecules. In solid tissues, oxygen, for example can diffuse radially from capillaries for only 150 to 200 μm.

When distances exceed this, cell death follows (Vincent et al., CANCER-Principles & Practice of Oncology, 5th Edition; Lippincott-Raven). Thus expansion of tumor masses beyond 2 mm in diameter depends on the development of an adequate blood supply. Many publications described the phenomenon of tumor angiogenesis. The observation that tumors could be implanted either into an avascular region, such as the cornea, or on a characteristically vascularised surface, such as the chick chorioalantoic membrane, and in each case form the ingrowths of new capillaries, suggested that tumors released diffusible activators of angiogenesis that could signal a quiescent vasculature to begin capillary sprouting (Hanahan and Folkman, Cell 1996; Vol. 86: 353-364).

In the past years, many inducers and inhibitors of angiogenesis have been identified by using a number of in vitro and in vivo bioassays such as the proliferation assay and the chemotaxis assay. The proliferation assay uses cultured capillary endothelial cells and measures either increased cell numbers or the incorporation of radiolabelled or modified nucleosides to detect cells in S-phase. In contrast, the chemotaxis assay separates endothelial cells and a test solution by a porous membrane disc, such that migration of endothelial cells across the barrier is indicative of a chemoattractant present in the test solution (Hanahan and Folkman, Cell 1996; Vol. 86: 353-364).

By the use of in vivo assays, such as implants in cornea, in vitro assays can be complemented. Several inducers and inhibitors of angiogenesis have been identified using these assays. The first to be detected was fibroblast growth factor and at about the same time, a secreted protein was identified by its ability to elicit vascular permeability called vascular endothelial growth factor or vascular permeability factor (VEGF) (Simpson-Haidaris and Rybarczyk, Annals of the New York Academy of Sciences 2001; Vol. 936: 406-425). It has been shown that both proteins (VEGF, FGF) are able to enhance capillary growth and thus, to be potent inducers of angiogenesis.

Recently, two related growth factors VEGF-B and VEGF-C have been identified and all three VEGF genes as well as FGF are widely expressed in normal adult organs of mice and humans, suggestive of roles in tissue homeostasis as well as angiogenesis. FGF and VEGF bind to receptors on endothelial cells that are transmembrane tyrosine kinases and thus are coupled through the signal transduction cascade to the cellular regulatory network (Brown et al., EXS 1997; Vol. 79: 233-269).

FGF and VEGF are commonly expressed in a wide variety of human and animal cancers and they have been detected at elevated levels in the urine and serum of a significant fraction of tumor-bearing patients (Simpson-Haidaris and Rybarczyk, Annals of the New York Academy of Sciences 2001; Vol. 936: 406-425). During the last decades, an increasing number of angiogenic inducers have been identified, but also first evidences of endogenous angiogenesis inhibitors have been presented. For example, researchers observed that a interferon and platelet factor-4 could inhibit endothelial cell chemotaxis and proliferation, respectively using the chemotaxis in vitro based assay and corneal pocket in vivo assay (Brouty-Boyé and Zetter, Science 1980; Vol. 208: 516-518).

The above paragraph summarizes the evidence that angiogenesis can be regulated both by inducers and inhibitors of endothelial cell proliferation and migration.

Today, there are clues suggesting that the balance of inhibitors and inducers governs the angiogenic switch. Several clues provide the basis for this hypothesis. The first clue comes from in vitro bioassays. As mentioned, FGF and VEGF can elicit a positive response from capillary endothelial cells using proliferation and chemotaxis assays. Experiments have demonstrated that increasing amounts of an inhibitor such as Thrombospondin-1 (TSP-1) blocks angiogenesis, suggesting that sufficient amounts of inhibitors overrule the signals of the positive activators, keep the angiogenic switch off (Dameron et al., Science 1994; Vol. 265: 1582-1584). The second clue comes from the study of human cancer cells, in which the restoration of p53 tumor suppressor function results in up-regulation of TSP-1, overruling both the angiogenesis inducers that the cells themselves synthesize as well as added FGF (Dameron et al., Science 1994; Vol. 265: 1582-1584). These and other clues suggest that changes in the balance of positive and negative signals mediate the “angiogenic switch” (see FIG. 2).

Knowing that vasculogenesis and angiogenesis, in particular neo-angiogenesis are key factors in tumor development, researchers were interested in interfering these processes with the aim of ultimately killing a tumor. Accordingly, more than 30 years ago, researchers postulated that in order to survive and grow, tumors require blood vessels, and that by cutting off blood supply, cancer could be starved into remission.

This revolutionary approach to cancer has evolved into one of the most exciting areas of scientific inquiry today. In the past decades, researchers have isolated the proteins and unravelled the processes that regulate angiogenesis (Ribatti, Angiogenesis 2008; Vol. 11: 3-10). In theory, each step of the angiogenic cascade is a potential target for cancer therapy. Numerous specific and unspecific inhibitors of endothelial proliferation and angiogenesis have been isolated and characterized. The National Cancer Institute (NCl) in the United States of America coordinates 19 potential angiogenesis inhibitors in clinical trials, whereas five of them are now in clinical phase 3 (NCl, official website of the National Cancer Institute). Two, quite promising inhibitors, angiostatin and endostatin, inhibit the proliferation of endothelial cells. The proliferation of pericytes, fibroblasts and tumor cells itself is not effected. Angiostatin showed 99% tumor inhibition in prostate cancer xenografts, whereas endostatin revealed tumor regression in several murine tumor models, such as Lewis-lung-carcinoma and B16F10 melanoma (O'Reilly et al., Cell 1994; Vol. 79: 315-328; O'Reilly et al., Cell 1997; Vol. 88: 277-285). Nowadays, leading pharmaceutical companies have recognized the promising topic and are working on developing new antibodies inhibiting angiogenesis activators such as VEGF, FGF or angiopoietins.

Several antibodies have been developed and tested with more or less great success. Avastin, for example, an antibody designed for inhibiting the vascular endothelial growth factor celebrated great success for its inhibiting effects in breast cancer and colon cancer patients. First studies are published, where angiogenesis inhibitors are used in gene therapy of malignant tumors. Besides that, in animal studies with specific angiogenic inhibitors, no side-effects have been reported so far (NCl, official website of the National Cancer Institute). The criticism that angiogenesis inhibitors can also inhibit important physiological functions, such as wound healing has been just partly reported for TNP-470. It has been shown that wound healing is delayed in mice (O'Reilly et al., Science 1999; Vol. 285: 1926-1928). Other inhibitors like endostatin or angiostatin did not show a negative influence towards wound healing or other physiological functions in mice.

In sum, a lot of efforts have been done to study angiogenesis. Researchers have isolated proteins that regulate angiogenesis. Several in vitro studies have been performed to study endothelial blood vessel formation. In the past decades, researchers realized that angiogenesis does also play a major role in tumor development. Early studies using implants in animals reported that angiogenic response, formation of blood vessels, induced by tumor tissue is more substantial and earlier than that induced by normal tissues or following a wound. Since, it has been shown that tumor growth is angiogenesis dependent, companies focused on developing new drugs against angiogenic growth factors to starve tumors into remission. For the purpose of monitoring tumor angiogenesis, in particular during therapy with anti-angiogenic drugs researchers tried and apply different imaging techniques, different blood vessel targets and dyes.

However, the so far applied imaging techniques do not allow in vivo imaging under real-time conditions. For example, Computer tomography (CT), positron emission tomography (PET), single-photon-emission computerized tomography (SPECT) and magnetic resonance imaging (MRI) are some of the classical non-invasive imaging techniques (see Table 1). These technologies allow the diagnosis of diseases, such as cancer, on the basis of anatomical, morphological and physiological changes. However, most of these established techniques lack sensitivity, reduced specific targeting and are incapable exhibiting functional changes on molecular basis and thus are not appropriate tools in basic-research, preclinical and translational applications. Therefore, diseases can be just diagnosed at late, morphologic visible points in time.

Other imaging techniques suffer from the deficiency that tumor material including vasculature has to be obtained from a subject.

The vasculature within a tumor generally undergoes active angiogenesis, resulting in the continual formation of new blood vessels to support the growing tumor. Such angiogenic blood vessels are distinguishable from mature vasculature in that angiogenic vasculature expresses unique endothelial cell surface markers, including the αᵥβ₃ integrin (Brooks et al., Cell 1994, Vol. 79:1157-1164; WO 95/14714) and receptors for angiogenic growth factors (Mustonen and Alitalo, J. Cell Biol. 1995, Vol. 129: 895-898; Lappi, Semin. Cancer Biol. 1995, Vol. 6: 279-288). Moreover, tumor vasculature is histologically distinguishable from blood vessel in general in that tumor vasculature is fenestrated (Folkman, Nature Med. 1995, Vol. 1: 27-31; Rak et al., Anticancer Drugs 1995, Vol. 6: 3-18. Thus, angiogenic vasculature is a particularly attractive target for targeting a tumor homing molecule.

Accordingly, αᵥβ₃ integrin, angiogenic growth factors such as VEGFs and their receptors are attractive targets for imaging tumor vasculature. U.S. Pat. No. 6,958,140 suggests ephrin-B2 as a blood vessel target for imaging, in particular, tumor vasculature. U.S. Pat. No. 6,051,230 suggests FGF as a target for imaging. EP 627 940 suggests ELAM-1-1, VCAM-1, ICAM-1, LAM-1 or an MHC Class II antigen. Citrin et al., Mol Cancer Ther. 2004, Vol. 4: 481-8 suggests endostatin. Bix et al., J Natl Cancer Inst. 2006, Vol. 98: 1634-1646 suggests endorepellin. Other authors suggest using a contrast agent for MPI, i.e., gadolinium, (Saban et al., BMC Cancer 2007, Vol. 7:219-238).

Moreover, so far no suitable interplay between imaging techniques, blood vessel markers and dyes was found that allows a non-invasive in vivo imaging of tumor vasculature or monitoring the therapeutic efficacy of an anti-angiogenic agent under real-time conditions in a subject. Accordingly, researchers stay with known techniques for imaging tumor vasculature and thus apply immunochemical techniques on cryosections of tumors (see Ninomiya et al., J. Surg. Res. 2009, Vol. 154: 196-262). Some researchers applied in vivo imaging of the fate of HUVECs supplemented by matrigel and implanted into mice by using a CD31 antibody (Bogdanov et al., Pharm. Res. 2007, Vol. 24: 1186-1192). These authors suggest using a CD31 antibody for in vivo imaging adoptive transfer of human endothelial cells in mice and using CD31 antibodies in determining the fate of human cells in a mouse model for bioengineering purposes and in drug development. Specifically, these authors want to follow the fate of human cells in a mouse and thus determined the specificity of an anti-CD31 antibody for human endothelial cells in a mouse. However, these authors did not use mouse tumor models to visualize the vasculation of a tumor by use of a labelled CD31 antibody. In addition, these authors suggest that non-invasive imaging could serve as an important adjunct to monitoring the fate of cell adoption and eventual acceptance in vivo. Accordingly, they further suggest that thus anti-CD31 antibody could be a potential imaging agent for imaging of adoptive transfer of human endothelial cells in mice and that these antibodies could assist in determining the fate of human cells for bioengineering purposes and in drug development. However, these authors do not suggest the use of anti-CD31 antibodies for in vivo monitoring the therapeutic efficacy of an anti-angiogenic agent in a subject.

Mitra and Foster, Neoplasia 2008, Vol. 10: 429-438, applied in vivo confocal fluorescence imaging to visualize the uptake and intratumoral distribution of the photosensitizer mono-L-aspartylchlorin-e6 (NPe6). A photosensitzer is used in photodynamic therapy. Photodynamic therapy is a third-level treatment for cancer involving three key components: a photosensitizer, light and tissue oxygen. Accordingly, Mitra and Foster investigated the distribution of the NPe6 photosensitizer and its kinetics. For that purpose they systemically administered NPe6 through the tail vein to the mouse EMT6 tumor model and visualized NPe6 with respect to the vasculature by using an Alexa Fluor 647-conjugated anti-mouse CD31 antibody. However, Mitra and Foster did not use a labelled anti-CD31 antibody to visualize tumor vasculature. They applied such an antibody to visualize the distribution of the NPe6 photosensitizer.

Runnels et al., Mol. Imaging. 2006, Vol. 5: 31-40, applied in vivo imaging in mice to visualize molecular expression of typical adhesion molecules of vascular endothelial cells. Accordingly, Runnels et al. administered monoclonal antibodies against, for example, CD31 (PECAM-1), E-selection, P-selection and VCAM-1 and investigated the temporal and spatial expression of these proteins. However, Runnels et al. did not visualize the tumor vasculature, since they did not use tumor models and were not interested in visualizing vasculature at all, but rather in visualizing expression of, for example, CD31.

JP 2009126864 discloses a blood vessel imaging agent that is capable of visualizing blood vessels in a membrane and which is a polymer combined with a target-directing group and group for detection.

In sum, despite the knowledge of various imaging techniques, various blood vessel targets and many dyes that are available for monitoring angiogenesis, in particular tumor angiogenesis, to the best of our knowledge, there is still a need to find appropriate means and methods for visualizing tumor angiogenesis and/or in vivo imaging tumor vasculature, in particular during and/or after therapy with therapeutic anti-angiogenic drugs. More specifically, there is still a need to provide non-invasive means and methods applying these means for in vivo imaging tumor vasculature in a subject as well as for imaging the therapeutic efficacy of an anti-angiogenic agent in a subject.

Hence, the technical problem of the present invention is to comply with the needs described above.

The present invention addresses this need and thus provides as a solution to the technical problem the embodiments concerning means, for example, tools and kits as well as methods and uses applying these means for in vivo imaging tumor vasculature and for in vivo monitoring the therapeutic efficacy of an anti-angiogenic agent in a subject by detecting fluorescence labelled anti-CD31 antibody. These embodiments are characterized and described herein, illustrated in the Examples, and reflected in the claims.

The inventors of the present application with the aim of studying tumor angiogenesis noninvasively, preferably under real-time conditions, in particular studying tumor angiogenesis noninvasively during and/or after therapy with a therapeutic anti-angiogenic agent applied different imaging techniques, blood vessel markers, means for binding these blood vessel markers and dyes facilitating the visualization of the binding of the means to these blood vessel markers. They also examined various imaging parameters with the aim to find a most appropriate imaging agent, dye and imaging technique to visualize tumor blood vessels, thereby imaging tumor vasculature.

They could achieve their aim and established and evaluated means and methods for visualization and quantification of tumor angiogenesis noninvasively. Specifically, they have found that, among the different imaging techniques, blood vessel markers, means for binding these blood vessel markers and dyes facilitating the visualization of the binding of the means to these blood vessel markers that have been tried, a fluorescence based monitoring method which applies an anti-CD31 antibody is most appropriate for noninvasively imaging tumor vasculature and/or monitoring the therapeutic efficacy of an anti-angiogenic agent in a subject by detecting a fluorescence labelled anti-CD31 antibody. It was surprising for the inventors that CD31 is a particularly suitable marker for angiogenesis during tumor development, since it could have been expected that tumor specific angiogenesis markers are more suitable.

Moreover, CD31 is normally found on endothelial cells, platelets, macrophages and Kupffer cells, granulocytes, T/NK cells, lymphocytes, megakaryocytes, osteoclasts, neutrophils. CD31 is also expressed in certain tumors, including epithelioid hemangioendothelioma, epithelioid sarcoma-like hemangioendothelioma, other vascular tumors, histiocytic malignancies, and plasmacytomas.

CD31 can also be found in the membranes of neutrophils, monocytes, bone marrow cells, immature lymphoid cells, myeloid hematopoietic cells and cells from leukemia and lymphoma patients, as well as in platelet and endothelial cell membranes.

Thus, it could have been expected that an anti-CD31 antibody would unspecifically bind to many cells/tissues, thereby generating too much background signal. However, the inventors surprisingly observed the opposite.

In sum, the developed method is feasible to monitor tumor angiogenesis specifically with high signal-to-background ratio. It allows the comparison of tumor vasculature and is able to monitor the ability and/or efficacy of anti-angiogenic agents to inhibit tumor blood vessel development and/or to exert a catabolic effect on tumor blood vessels.

Accordingly, in a first aspect the present invention relates to a non-invasive method of in vivo imaging tumor vasculature in a subject comprising detecting a fluorescence labelled anti-CD31 antibody. The methods and uses described herein allow real-time imaging, i.e., vasculature, in particular the tumor vasculature, is directly visualized so that no delay in developing pictures or the like is required. Accordingly, any change in the vasculature, in particular tumor vasculature, may be directly (online) visualized. An advantage of real time imaging is that an effect of, for example, an anti-angiogenic agent may be monitored shortly after the administration of such an agent.

The term “in vivo imaging tumor vasculature” also includes in vivo imaging or in vivo monitoring angiogenesis occurring during tumor development. It also includes in vivo imaging or in vivo monitoring blood vessels, in particular tumor blood vessels.

Specifically, the method applied is based on a fluorescence labelled anti-CD31 antibody that has proved to be target specific and visualizes blood vessels properly.

As has been demonstrated by the inventors, CD31 is a particular useful marker for visualizing tumor vasculature, since CD31 is a marker expressed during angiogenesis, in particular during angiogenesis in tumor development.

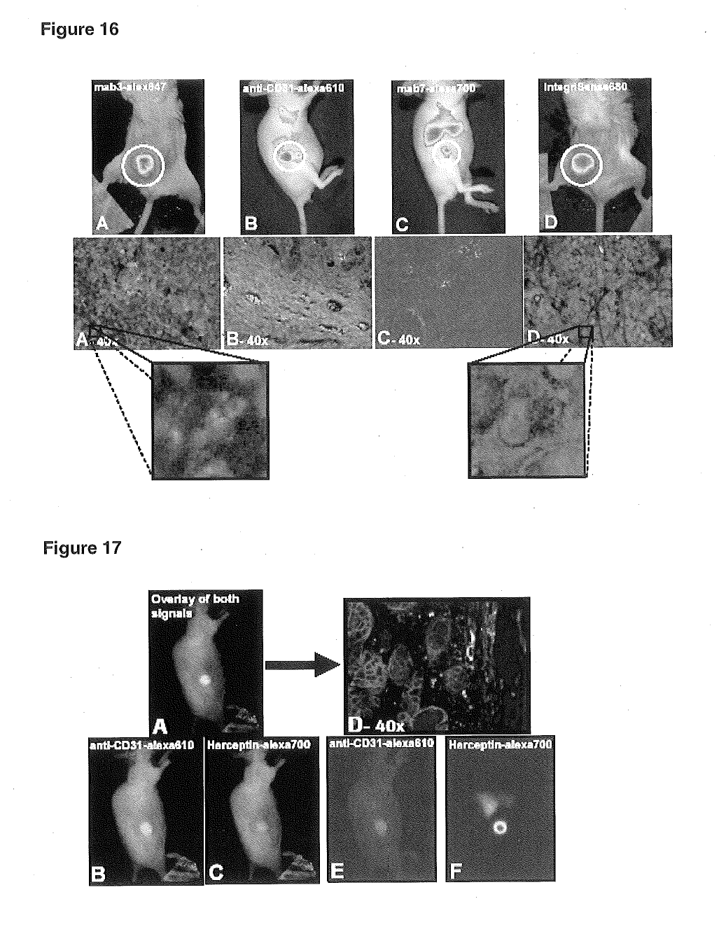

More specifically, different targeting agents, antibody-based or non-peptide molecules were examined for imaging blood vessel growth and distribution. All agents have key roles in angiogenesis and are important targets for drug development. They face more stringent design criteria than in vitro reagents. The basic underlying difficulty is to design them with high target to background ratios. For example, there should be minimal non-specific tissue extravasations, internalization by macrophages, or renal or hepatic elimination but should yield high local concentrations at the region of interest. For these reasons, mab3-alexa647, anti-CD31-alexa610 and mab7-alexa700 underlying a specific labelling design though, multiple copies of the alexa labels were used for a single antibody (in a ratio of approx. ⅓). The fourth imaging agent, IntegriSense 680 is a targeted fluorescence imaging agent comprising a potent, selective non-peptide small molecule integrin αᵥβ₃ antagonist and NIR fluorochrome. It has been developed for the visualization of integrin αᵥβ₃ expression, a cell-adhesion molecule that contributes to endothelial cell migration by mediating cell-extracellular-matrix interactions in neovasculature as well as in tumor cells. Imaging results with Mab3-alexa647 revealed a strong and tumor concentrated signal, however, Nuance imaging studies indicated that the antibody is associated with tumor cells, but blood vessels could not be detected (see FIG. 16A). Is has been shown that the Mab3-growth-factor complex is incorporated by the tumor in monkey retinal sections. In contrast, anti-CD31-alexa610 imaging exhibited a regular distribution of the antibody in the whole mouse body. This may be explained by the monoclonal antibody used (antimouse-CD31) and the minimal expression of mouse CD31. Histological examinations showed well defined outlines of blood vessel capillaries (see FIG. 16B). The third imaging reagent, mab7-alexa700 and its angiogenic growth factor target, seems to circulate through the whole blood system ending up in lung and liver. Nuance imaging could detect only weak signal intensities and small amounts of the antibody in the murine stroma. No blood vessels were visible (see FIG. 16C). IntegriSense680, the non-peptide small molecule that is considered to be able to visualize neovasculature is not an appropriate imaging agent for monitoring angiogenesis in the xenograft used. In vivo, it gives a strong signal with low target-to-background ratio. However, this signal was generated by targeting tumor cells instead of the desired blood vessels (see FIG. 16D).

To this end, the inventors observed that only an anti-CD31 antibody labels vascular endothelial cells specifically with high signal-to-background ratio.

Any suitable imaging system allowing the detection of fluorescence or near-infrared fluorescence labelled structures, for example, organs or tumors, can be applied in the kits, methods and uses of the invention.

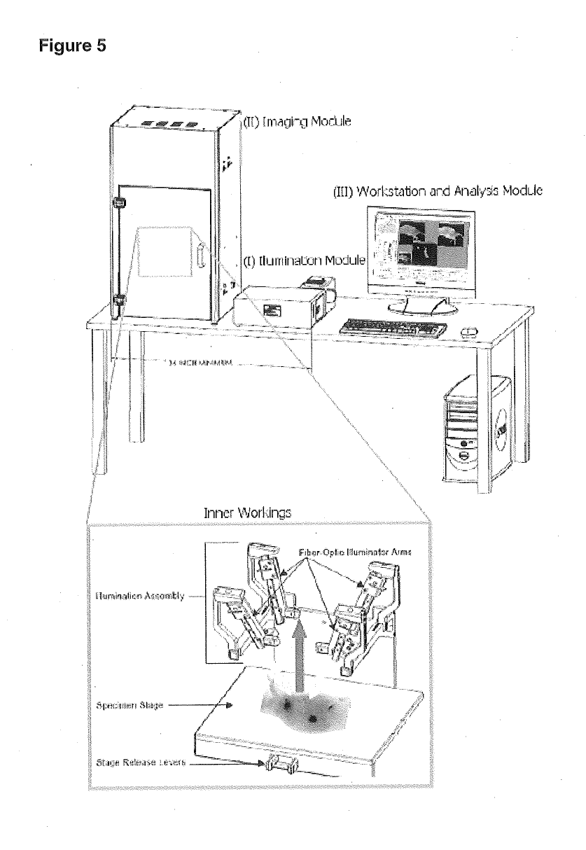

A preferred imaging system is the MAESTRO system described herein (see FIG. 5) which allows target specific in vivo live imaging to examine tumor vasculature including angiogenesis in a tumor. Besides this, it is possible to determine target specific signals in just one mouse at the same time. The program is able to separate the two emission spectra and visualize both reagents in different colours. Furthermore, the signal intensities can be compared and allow conclusions about target concentrations and distributions. Herceptin, a humanized monoclonal antibody, which binds to the HER2 receptor was labelled with alexa700 and injected together with anti-CD31-alexa610 in equal concentrations to examine the different expression patterns and the sensitivity of the imaging system (see FIG. 17). The fluorescence signals could be separated and the comparison of signal intensities revealed that the CD-31-signal is hundred-fold weaker than the one for Herceptin-alexa700 (see FIGS. 17E-F). These results were confirmed by ex vivo Nuance images as described herein. Herceptin, concentrated on tumor cell surfaces, was much more present than mouse CD31+ endothelial cells representing blood vessel capillaries.

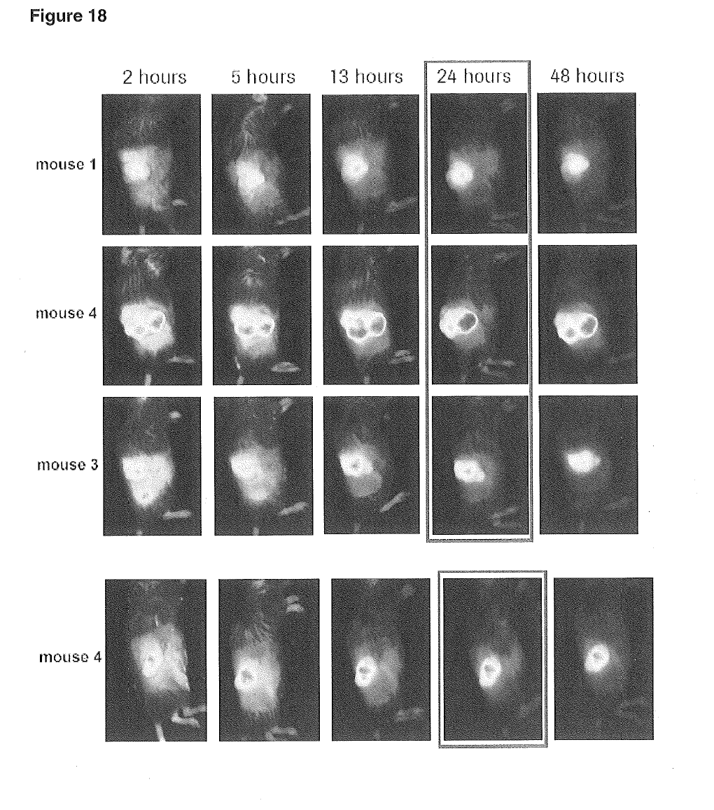

For ideal imaging conditions, a high target-to-background ratio is desired. Diminished doses of circulating imaging reagents and marginal unspecific bindings are necessary for best imaging results. In contrast, the target specific concentration within the tumor should be at the maximum. The imaging results for IntegriSense680 demonstrated a decrease of the background signal progressively over time. Imaging figures of 2 hrs and 5 hrs post-reagent-injection revealed high background signals caused by circulating fluorescent agents or unspecific bindings (see FIG. 18). Later in time, these probes may be metabolized and subsequently eliminated.

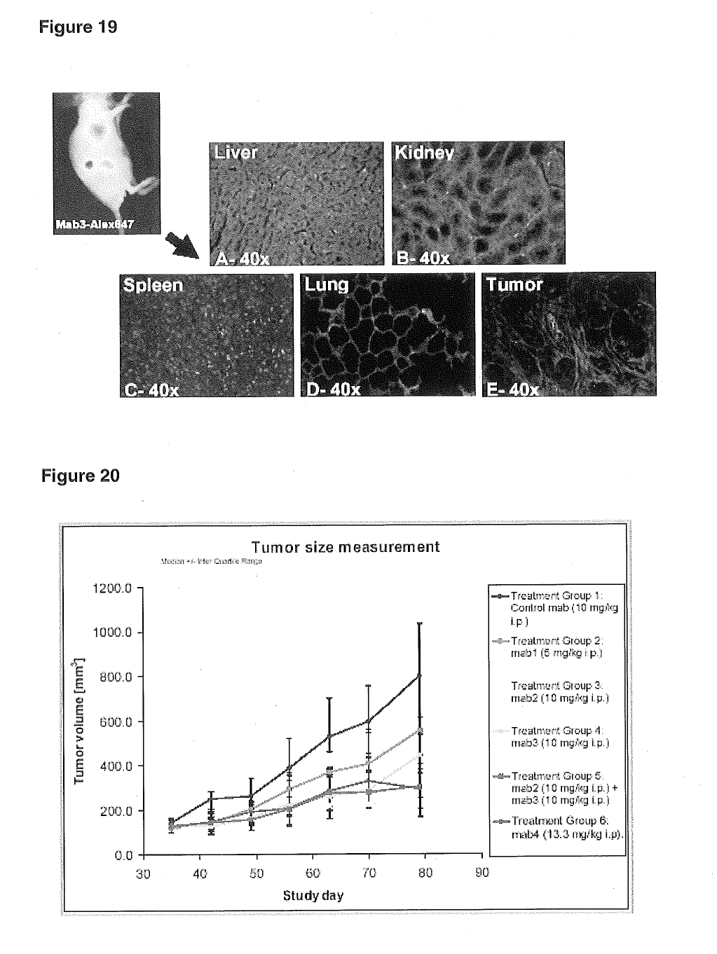

The MAESTRO imaging system is a planar fluorescence-reflecting-imaging system and therefore, tissue penetration of near-infrared radiation is limited. The fluorescence signal intensities are depending on their location (depth) in the mouse tissue. Hence, signals of imaging reagents in organs like liver, spleen, kidney and lung relative to signals in upper tissue sections like a subcutaneous tumor, can not be exactly quantified. Mab3-alexa647 signal intensities, obtained from in vivo live imaging, in tumor and excretory organs did not correlate with the signal distribution in ex vivo studies with the Nuance system. While in vivo studies exhibited highest agent concentrations in the tumor, fluorescence examination of explanted organs revealed higher, or at least similar, concentrations in liver, kidneys and spleen (see FIG. 19). These results are in concordance with the theory that signal intensity is highly dependent on the tissue depth of the imaging agents.

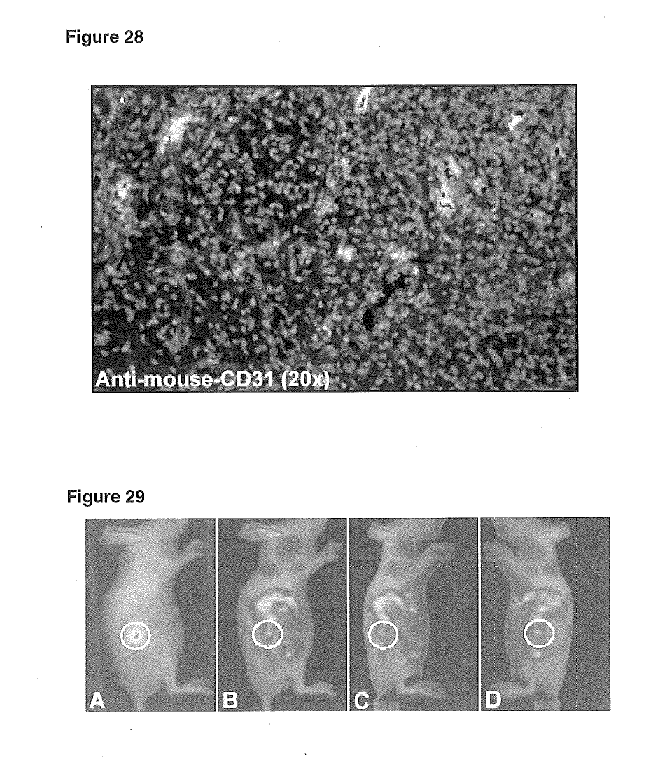

In sum, several imaging agents, specific for angiogenic targets, have been evaluated for monitoring tumor angiogenesis in vivo and noninvasively using in vivo fluorescence imaging, preferably near-infrared imaging. Imaging probes can be evaluated in vivo with the MAESTRO system and, optionally, as ex vivo in formalin fixed paraffin-embedded tumor sections to test the sensitivity and specificity of these markers. The identification of endothelial cells and vascularity of tumors are a prerequisite for subsequent studies. Only the anti-CD31 marker labels vascular endothelial cells specifically with high signal-to-background ratio (see FIG. 28). It also allows the ex vivo visualization of single blood vessels as a starting point for the quantification process.

The present invention also contemplates that, in addition to in vivo imaging tumor vasculature or in vivo monitoring the therapeutic efficacy of an anti-angiogenic agent in a subject as described herein, the location of the labelled anti-CD31 antibody can be monitored/imaged in a subject by detecting the labelled antibody with any of the imaging techniques described herein.

This additional aspect provides an overview on the location of the antibody. For example, it can be monitored as to whether the antibody is also in the blood, in particular in the serum, in the kidneys, bladder, etc. Accordingly, one can also judge on the specificity, half-life, distribution, etc. of the antibody.

In the claimed methods and uses the antibody is administered parenterally, preferably intravenously to a subject, preferably at different time points during therapy and is imaged in vivo by a fluorescence imaging system, preferably by a near-infrared fluorescence imaging system such as a fluorescence reflectance imaging (FRI) system or by a fluorescence-mediated tomography (FMT) system.

An imaging system for NIR fluorescence measurement useful in the practice of this invention typically includes three basic components: (1) a near infrared light source, (2) a means for separating or distinguishing fluorescence emissions from light used for fluorochrome excitation, and (3) a detection system.

The light source provides monochromatic (or substantially monochromatic) near infrared light. The light source can be a suitably filtered white light, i.e., bandpass light from a broadband source. For example, light from a 150-watt halogen lamp can be passed through a suitable bandpass filter commercially available from Omega Optical (Brattleboro, Vt.). In some embodiments, the light source is a laser. See, e.g., Boas, D. A., et al., Proc. Natl. Acad. Sci. USA 1994, Vol. 91: 4887-4891; Ntziachristos, V., et al., Proc. Natl. Acad. Sci. USA 2000, Vol. 97: 2767-2772; Alexander, W., J. Clin. Laser Med. Surg. 1991, Vol. 9: 416-418.

A high pass filter (700 nm) can be used to separate fluorescence emissions from excitation light. A suitable high pass filter is commercially available from Omega Optical.

In general, the light detection system can be viewed as including a light gathering/image forming component and a light detection/image recording component. Although the light detection system may be a single integrated device that incorporates both components, the light gathering/image forming component and light detection/image recording component can be separate.

Any suitable light detection/image recording component, e.g., charge coupled device (CCD) systems or photographic film, can be used in the invention. The choice of light detection/image recording will depend on factors including type of light gathering/image forming component being used. Selecting suitable components, assembling them into a near infrared imaging system, and operating the system is within ordinary skill in the art.

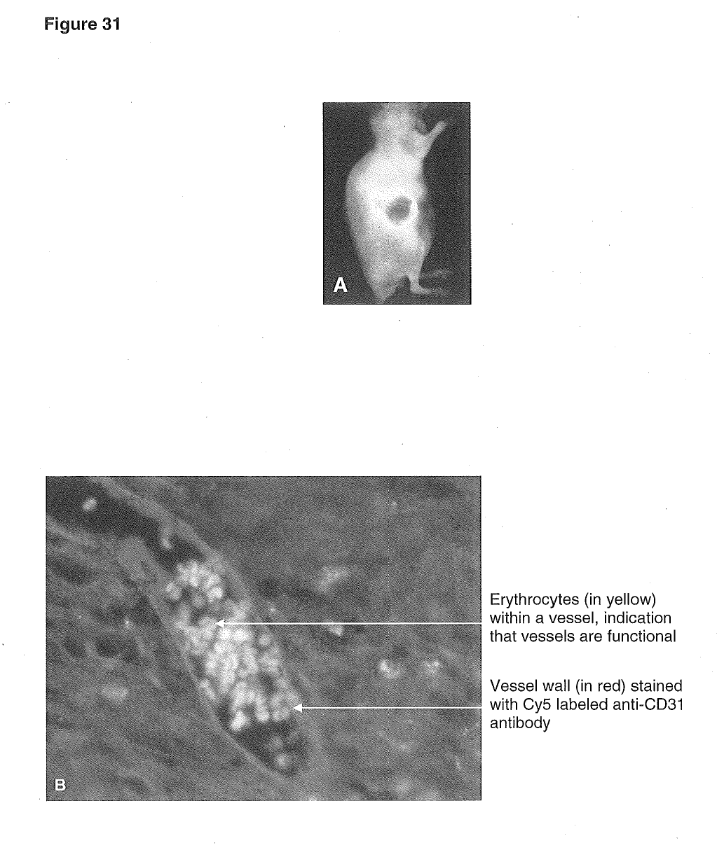

The in vivo imaging techniques applied in the present invention also include the FMT technology (fluorescence molecular tomography), a laser based three-dimensional imaging system that provides non-invasive, whole body, deep tissue imaging in small animal models and generates 3D reconstruction of fluorescence sources and/or allows measurement of fluorescence concentrations of fluorescence labelled structures due to binding of the fluorescence labelled anti-CD31 antibody applied in the present invention. The system allows determining the real distribution of imaging probes. Planar, two-dimensional imaging (see FIG. 29A) reveals highest antibody concentration within the tumor region, whereas 3D imaging studies with the FMT system allows improved assessment of the antibody distribution in the mouse body (see FIGS. 29B-D and FIG. 31). Therefore, quantification of fluorescence signal intensities can be analyzed in a more realistic approach.

When performing the methods and uses of the invention, it is preferred that the fluorescence labelled anti-CD31 antibody is to be administered to the subject prior to detecting the antibody, i.e., the signals from the fluorescence labelled anti-CD31 antibody. As mentioned above, it is then envisaged that the antibody is to be administered at different time points during therapy and, optionally, after the therapy for the purpose of control, exclusion of recurrence of another tumor at the same location and/or at a location different from the first location. Also, the antibody may be administered prior to therapy for the purpose of visualizing the tumor vasculature before therapy and/or to visualize the tumor prior to monitoring the therapeutic efficacy of an anti-angiogenic agent in order to then assess whether the anti-angiogenic agent is therapeutically effective

An example of a near-infra red fluorescence imaging system is the MAESTRO system described in the appended Examples. Accordingly, the MAESTRO system is a preferred system that may be applied in the kits, methods and uses of the present invention.

The MAESTRO system is a planar fluorescence-reflecting-imaging system that allows a noninvasive in vivo fluorescence measurement. In this multispectral analysis, a series of images are captured, at specific wavelengths. The range of wavelengths captured should cover the expected spectral emission range of the label present in the specimen. The result will be a series of images called “image cube” and it is the data within this series of images that is used to define the individual spectra of both auto-fluorescence and specific labels. Many labels of biological interest have emission spectra that are so similar that separation using expensive narrow band filters is difficult or impossible. A single long pass emission filter replaces a large collection of emission filters. In addition to the natural auto-fluorescence of the skin, fur, sebaceous glands, there is also distinct auto-fluorescence from commensal organisms (fungi, mites, etc.) and ingested food (chlorophyll). Multispectral analysis is able to separate all of these signals from the specific label applied to the specimen through the mathematically disentanglement of the linear signal mixture (unmixing) of the emitted fluorescent lights as long as the emission spectrum of the desired signal and of the auto-fluorescence are known.

Measurement with the MAESTRO system works as follows: The illumination module is equipped with a xenon lamp (Cermax) that excites white light. Through a downstream connected excitation filter (chosen by the experimenter), the light is delimitated to a, for the experiment, desired wavelength range and conducted via an optical fiber into the imaging module. In here, the restricted light is partitioned into four optical fibers that illuminate the anesthetized test animal. The MAESTRO system chooses the optimal exposure time automatically, so that there is no risk of overexposure. The emitted fluorescence light of the activated fluorescent probe is selected with an emission filter (see Table 1) and conducted through a liquid crystal (LC) to a high sensitive, cooled CCD-camera. The liquid crystal enables the camera a selective picture recording of a specific wavelength. The wavelength measurement range depends on the selected filter set (blue, green, yellow, red, deep red, NIR) and pictures are recorded in steps of 10 nm. The spectral information of each single picture is combined in one “picture package” that is called “image cube”.

TABLE 1
Maestro filter sets.

| Maestro | | Excitation | Emission | Acquisition |
|---|---|---|---|---|
| Filter Set | Part # | Filter | Filter | Settings* |
| Blue | M-MSI-FLTR- | 445 to 490 nmm | 515 nm | 500 to 720 in |
| | BLUE | | longpass | 10 nm steps |
| Green | M-MSI-FLTR- | 503 to 555 nm | 580 nm | 550 to 800 in |
| | GREEN | | longpass | 10 nm steps |
| Yellow | M-MSI-FLTR- | 575 to 605 nm | 645 nm | 630 to 850 in |
| | YELLOW | | longpass | 10 nm steps |
| Red | M-MSI-FLTR- | 615 to 665 nm | 700 nm | 680 to 950 in |
| | RED | | longpass | 10 nm steps |
| Deep Red | M-MSI-FLTR- | 671 to 705 nm | 750 nm | 730 to 950 in |
| | DEEP-RED | | longpass | 10 nm steps |
| NIR | M-MSI-FLTR- | 710 to 760 nm | 800 nm | 780 to 950 in |
| | NIR | | longpass | 10 nm steps |

The analysis with the MAESTRO system works as follows:

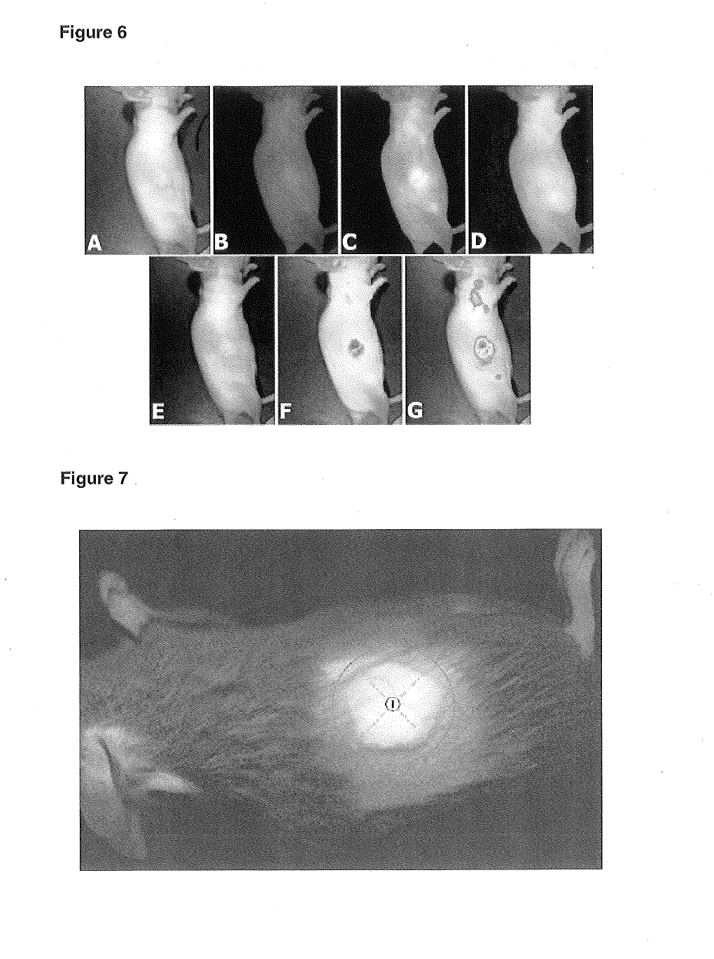

Each recording compose of 12 bit black-and-white pictures that can be illustrated in 4096 different gray scales and therefore it is possible to discriminate between smallest differences in emission intensities. In contrast, the human eye is able to distinguish between 30-35 grey scales. Those values for the emission intensities (grey scales) are plotted against the wavelength range and as a result, we obtain the emission spectra of each probe and the tissue auto-fluorescence. The software subdivides the three fundamental colours (red, green, blue) to the wavelength range used for the imaging cube whereby the black-and-white pictures turn into coloured image (RGB image, see FIG. 6B). Out of these acquired multispectral information the system is able to differentiate between injected probes and auto-fluorescence of any source. The program is using a spectral library, where the single spectra of each pure probe and the spectra acquired by imaging the study animals (Balbc/nude or Scid Beige mice) without any injection (mouse auto-fluorescence). By knowing the exact spectra of the pure imaging and of the auto-fluorescence, the system is able to filter the whole image for the desired spectra and assign a colour to each of them. The originated image (unmixed composite image) shows the present spectra in different colours. To visualize the intensity distribution of the probe signal, it is possible to illustrate the signal in false colours, whereas low intensities are blue and regions of high intensities are red. Besides that, one can define a detection limit for the signal intensity of the probe, which allows reducing the signal of circulating probes and unspecific bindings.

Comparison and quantification with the MAESTRO system works as follows:

The MAESTRO's ability to compare fluorophore regions of an image makes it easy to compare the tumor fluorescent signal intensities during therapy. The program provides tools for the comparison of different signal intensities in tumor regions (compared images). Since all images are taken at optimal exposure times, they differ depending on the strength of signals. For a reliable comparison, the pictures are standardized to one exposure time, resulting in an illustration of differences in signal intensities. By manually drawing and modifying measurement regions, signal intensities can be quantified in intensity values. Once a measurement area is selected around the tumor, it can be cloned and moved to the next image to be compared with. Each region is calculated in pixels and mm2 based on the current settings (stage height and binning). As a result, it gives information about the average signal, total signal, max. signal and average signal/exposure time (1/ms) within the created measurement area (see FIG. 7).

An in vivo fluorescence imaging system, in particular a near-infra red fluorescence imaging system which is preferably applied in the methods, uses and kits of the present invention allows the comparison and quantification of the in vivo signals and thus, for example, does not only allow in vivo imaging of vasculature, in particular tumor vasculature, but also the effects of an anti-angiogenic agent can be revealed and compared by the increase or decrease of the fluorescence labelled anti-CD31 antibody signal over time. Namely, an increase or decrease of blood vessels can be visualized by the binding (or non-binding) of the labelled anti-CD31 antibody to CD31, i.e., the less angiogenesis/blood vessels, the less is the signal provided by the antibody or, likewise, the more blood vessels, the more is the signal provided by the antibody.

Ex vivo experiments have been applied to confirm the in vivo data. In particular, tumors were explanted, fixed and embedded in paraffin to examine the tumor sections with a fluorescence microscope.

Accordingly, in a preferred aspect of the present invention the kits, methods and uses described herein may include a step which includes the confirmation of the in vivo data by any technique suitable for this purpose, for example, by immunochemistry, immunohistochemistry, histological techniques, for example, issue staining, or mRNA expression profiling.

Specifically, for tissue staining, tumors are explanted, sections are prepared, fixed, embedded in paraffin and stained with, for example, hematoxylin-eosin as is described in more detail in the appended Examples. So-stained tumor sections can be imaged ex vivo by, for example, the Nuance® system.

The multispectral imaging technique with the Nuance camera acquires high sensitive images on paraffin or cryosections and allows quantitative and sensitive measurements of subtle differences in spectral emissions. Nuance has two main advantages. First, it is able to do brightfield imaging and it can also act as an excellent colour camera. Secondly, in fluorescence microscopy, in samples which are auto-fluorescent, like most tissue sections, Nuance is separating tissue auto-fluorescence from signals of interest by using a filter set and multispectral analysis. The Nuance system is also described in the appended Examples and is a preferred embodiment applied in the kits, methods and uses of the invention.

As an example for tumor explanation, animals are sacrificed and tumors are explanted according to committed guidelines (GVSolas, Felasa, TierschG). After sacrificing the animals, tumors are explanted using, for example, a scalpel and transferred into an embedding cassette or flash frozen in liquid nitrogen with isopentan for immunohistochemical applications and without isopentan for mRNA/PCR applications.

As an example for fixation, explanted tumors are enclosed in an embedding cassette and are incubated under continuous agitation in formalin for preferably approximately 24 hours. Thereafter, formalin is discarded and tumors washed with dest. water followed by dehydration of the tumors and penetration of paraffin. All the incubation steps are carried out using the Tissue Tek® VIP Vacuum Infiltration Processor.

After paraffin penetration, tumors are embedded in liquid paraffin with the Tissue Tek® Paraffin embedding station to a final histological block. Paraffin sections in a range of 2-8 μm of thickness are obtained from these blocks using a microtome. They are cut, uptaken on glass slides, air dried over night at 37° C. followed by de-paraffinization and HE-staining (see Example 4.10 and Tables 6 and 7). Slides are examined with the Nuance system.

In addition, the present invention also provides in a preferred aspect a method to quantify fluorescence labelled blood vessel areas in tumor sections. This quantification may be applied as a further step in the methods and uses of the present invention.

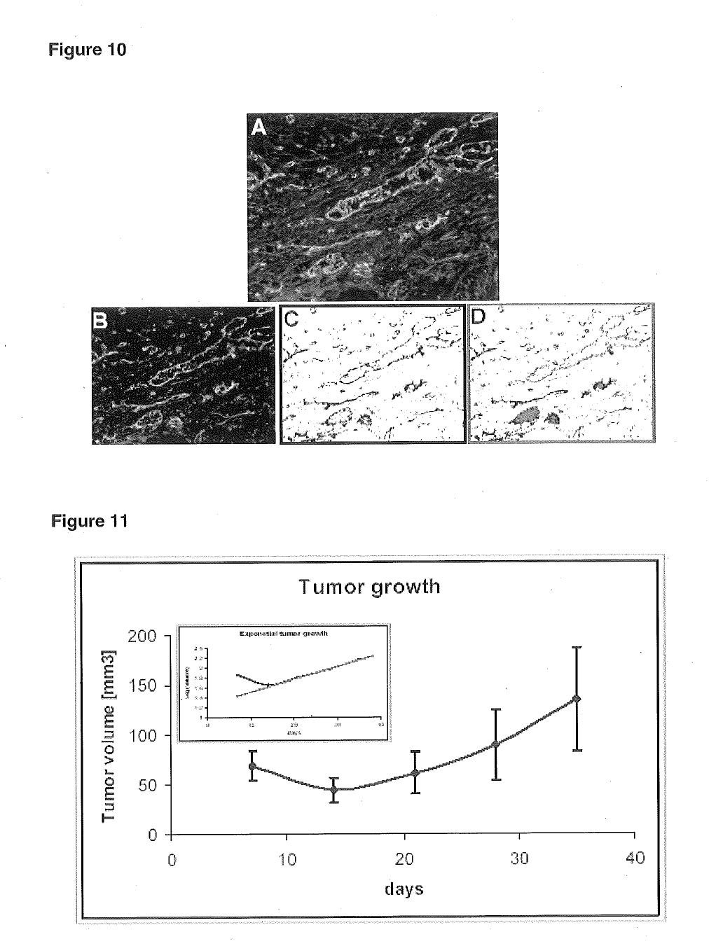

Specifically, tumor sections are prepared as described herein above in the context of tissue staining of tumors. Accordingly, the quantification of blood vessel density in paraffin sections is carried out using a custom written MATLAB script which separates the anti-CD31 signal by using a k-Means-clustering (see FIG. 10B). The filtered specific signal is then transformed into a binary image and “eroded” to remove disturbing signals (erosion function). After reversing the picture (see FIG. 10C), it is possible to manually define the regions of blood vessel appearance. The program marks the region of interest in red (see FIG. 10D) and calculates the percentage of blood vessel regions within the recorded picture.

This blood vessel quantification tool allows the percentage of blood vessel area per slide. It is envisaged that this tool can be applied in such a way that the system should identify and calculate blood vessel areas automatically by artificial intelligence. The system will be able to discriminate between blood vessel areas and unspecific signals and calculates the embedded blood vessel areas.

As regards immunohistochemistry, an unlabelled anti-CD31 antibody is parenterally injected, preferably intravenously, tumors are explanted, preferably at around 24 hours post-injection of the antibody, flash frozen in, for example, liquid nitrogen and isopentan and sectioned. Unlabelled anti-CD31 antibody is identified by using a secondary antibody directed against the Ig subtype of the unlabelled anti-CD31 antibody, for example, goat anti-mouse IgG. This technique is described in more detail in the appended Examples and is a preferred embodiment of the kits, methods and uses of the present invention.

As an example for immunohistochemistry, tumor blood vessels are visualized through the whole tumor section, an unlabelled endothelial antibody (anti-mouse-CD31 from mouse, hamster, rat, etc.) is injected into a subject. For example, in a mouse an unlabelled anti-mouse-CD31 from hamster is injected in a concentration of 50 μg/mouse. Tumors are explanted at 24 hours post-injection and sectioned after flash freezing in liquid nitrogen and isopentan. The unlabelled vessel marker is identified using a secondary antibody (goat anti-hamster IgG) alkaline phosphatase system (see FIG. 9). A slide scanner allows the visualization of the whole tumor section.

For Immunohistochemistry applications, tumors are flash frozen immediately after excision in Isopentan and liquid nitrogen and held at −20° C. until sectioning. For example, 10 μm tumor cryosections are cut at the optimum cutting temperature of −18° C. with the Cryostat (2800 Frigocut), fixed with acetone for one minute, air dried and used for Immunohistochemistry application.

The immunostaining procedure and the slight scan are described in detail in the pending examples.

With respect to mRNA expression profiling, tumors are explanted and flash frozen in, for example, liquid nitrogen (without isopentan). Tumors are lysed, homogenised and RNA is prepared according to methods commonly known in the art and also shown in the appended Examples. Afterwards, cDNA is prepared and analysed qualitatively and/or quantitatively for the expression of an angiogenic growth factor such as FGF, VEGF-A, VEGF-B, VEGF-C, VEGF-D, their receptors such as VEGFR1 or VEGFR2 and/or Angiopoietin. As a loading control in a quantitative expression analysis a typical housekeeping gene such as β-actin or GAPDH can be used.

A preferred embodiment of mRNA expression profiling is described in more detail in the appended Examples. It can be used to monitor the effects of a therapeutic anti-angiogenic agent such as those described herein by, for example, determining the presence and/or level of expression of genes involved in angiogenesis such as VEGF-A, VEGF-B, VEGF-C, VEGF-D, VEGFR1, VEGFR1 and/or Ang2 in a tumor sample. Accordingly, mRNA expression profiling is a preferred embodiment of the kits, methods and uses of the present invention that may be applied in a further, i.e., additional step.

The antibody applied in the methods and uses of the present invention is labelled with a fluorescence label including near-infrared fluorescence label. Accordingly, when used in the context of the kits, methods and uses of the invention, the term “fluorescence” includes fluorescence labels commonly known in the art or as described herein and also includes near-infrared fluorescence labels, the latter being preferred in the context of the present invention. Fluorescence labelling is accomplished using a chemically reactive derivative of a fluorophore. Common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide. Reaction of any of these reactive dyes with another molecule results in a stable covalent bond formed between a fluorophore and a labelled molecule. Accordingly, the antibody of the present invention can be labelled with the aforementioned fluorescence labels. Other suitable fluorescence labels envisaged by the present invention are Alexa Fluors, Dylight fluors and ATTO Dyes. Likewise, fluoresecent proteins such as GFP, YFP or CFP may be used. Also, activatable fluorescent dyes which are activated by pH changes or voltage, temperature are envisaged to be used. It is also envisaged that the labelled anti-CD31 antibody comprises more than one fluorescence or near-infrared fluorescence label, for example, 2, 3, 4, 5, 6, 7, 8, 9 or 10. Likewise, it is envisaged that 2, 3, 4, 5, 6, 7, 8, 9 or 10 different fluorescence or near-infrared fluorescence labelled anti-CD31 antibodies are applied in the kits, methods and uses of the invention. These different antibodies bind to CD31, however, may bind to different epitopes.

In the alternative, the anti-CD31 antibody of the invention may be indirectly labelled. For example, an unlabelled anti-CD31 antibody is labelled (due to its binding to the constant region of the unlabelled antibody) by a second antibody directed against the unlabelled antibody, wherein the second antibody delivers the fluorescence signal.

As mentioned, the antibody applied in the kits, uses and methods of the invention can also be labelled with a near-infrared (NIR) fluorescence label. NIR fluorescence labels with excitation and emission wavelengths in the near infrared spectrum are used, i.e., 640-1300 nm preferably 640-1200 nm, and more preferably 640-900 nm. Use of this portion of the electromagnetic spectrum maximizes tissue penetration and minimizes absorption by physiologically abundant absorbers such as hemoglobin (<650 nm) and water (>1200 nm). Ideal near infrared fluorochromes for in vivo use exhibit:

(1) narrow spectral characteristics,
(2) high sensitivity (quantum yield),
(3) biocompatibility, and
(4) decoupled absorption and excitation spectra.

Various near infrared (NIR) fluorescence labels are commercially available and can be used to prepare probes according to this invention. Exemplary NIRF labels include the following: Cy5.5, Cy5 and Cy7 (Amersham, Arlington Hts., IL; IRD41 and IRD700 (L1-COR, Lincoln, Nebr.); NIR-I, (Dejindo, Kumamoto, Japan); LaJolla Blue (Diatron, Miami, Fla.); indocyanine green (ICG) and its analogs (Licha, K., et al., SPIE-The International Society for Optical Engineering 1996; Vol. 2927: 192-198; U.S. Pat. No. 5,968,479); indotricarbocyanine (ITC; WO 98/47538); and chelated lanthanide compounds and SF64, 5-29, 5-36 and 5-41 (from WO 2006/072580). Fluorescent lanthanide metals include europium and terbium. Fluorescence properties of lanthanides are described in Lackowicz, J. R., Principles of Fluorescence Spectroscopy, 2nd Ed., Kluwer Academic, New York, (1999).

The antibody of the present invention is preferably labelled by a NIR fluorescence label selected from the group of Cy5.5, Cy5, Cy7, IRD41, IRD700, NIR-I, LaJolla Blue, indocyanine green (ICG), indotricarbocyanine (ITC) and SF64, 5-29, 5-36 and 5-41 (from WO 2006/072580), more preferably said antibody is labelled with a NIRF label selected from the group of Cy5.5, Cy5 and Cy7.

The methods used for coupling of the NIR fluorescence labels are well known in the art. The conjugation techniques of NIR fluorescence labels to an antibody have significantly matured during the past years and an excellent overview is given in Aslam, M., and Dent, A., Bioconjugation (1998) 216-363, London, and in the chapter “Macromolecule conjugation” in Tijssen, P., “Practice and theory of enzyme immunoassays” (1990), Elsevier, Amsterdam.

Appropriate coupling chemistries are known from the above cited literature (Aslam, supra). The NIR fluorescence label, depending on which coupling moiety is present, can be reacted directly with the antibody either in an aqueous or an organic medium. The coupling moiety is a reactive group or activated group which is used for chemically coupling of the fluorochrome label to the antibody. The fluorochrome label can be either directly attached to the antibody or connected to the antibody via a spacer to form a NIR fluorescence label conjugate comprising the antibody and a NIR fluorescence label. The spacer used may be chosen or designed so as to have a suitably long in vivo persistence (half-life) inherently.

A subject when used herein includes mammalian and non-mammalian subjects. “Mammal” for purposes of treatment refers to any animal classified as a mammal, including human, domestic and farm animals, nonhuman primates, and any other animal that has mammary tissue. A mammal includes human, rodents such as mouse, rat or rabbit, dog, cat, chimpanzee etc., with human being preferred. A subject also includes human and veterinary patients. A non-mammalian subject is, for example, a chicken, a duck or a zebra fish. In preferred aspects of the methods and uses of the invention, the subject is to be treated with an anti-angiogenic agent.

Moreover, the present inventors applied the methods and uses described herein to monitor the therapeutic efficacy of anti-angiogenic agents towards tumor vasculature including angiogenesis in a subject and found that it is also appropriate for this purpose.

In particular, the methods and uses of the invention allow the comparison and quantification of in vivo signals and thus, the effects of anti-angiogenic agents can be revealed and compared by the increase or decrease of the fluorescence labelled anti-CD31 antibody signal over time

More particularly, the increase or decrease of anti-CD31 antibody fluorescence signals in a subject during and/or after treatment with an anti-angiogenic agent is determined and compared to anti-CD31 antibody fluorescence signals before treatment with an anti-angiogenic agent. This comparison allows to judge on the therapeutic effect of the anti-angiogenic agent.

Accordingly, the methods and uses of the present invention provide data and/or information regarding the correlation between the change of tumor volumes and the change of tumor vasculature, thereby allowing to monitor the therapeutic effect of an anti-angiogenic agent in a subject.

The so-obtained data and/or information may, in addition, be confirmed by histological applications such as tissue staining, immunochemistry or immunohistochemistry as described herein and may thus be performed as an additional step of the methods and uses of the present invention.

Alternatively or in addition, the so-obtained data and/or information may be confirmed by mRNA expression profiling so as to reveal the effects of an anti-angiogenic agent and may thus be performed as an additional step of the methods and uses of the present invention. In essence, the methods and uses of the present invention are suitable to monitor angiogenesis during tumor development, preferably with high signal-to-background ratio. It thus allows the comparison of tumor vasculature and is able to reveal the different ability or efficiency of anti-angiogenic therapeutic agents to inhibit tumor blood vessel development and/or to catabolise tumor blood vessels.

The term “antibody” as used herein refers to an immunoglobulin or fragment thereof, and encompasses any polypeptide comprising an antigen-binding fragment or an antigen-binding domain. The term includes but is not limited to polyclonal, monoclonal, monospecific, polyspecific, non-specific, humanized, human, single-chain, chimeric, synthetic, recombinant, hybrid, mutated, grafted, and in vitro generated antibodies. Unless preceded by the word “intact”, the term “antibody” includes antibody fragments such as Fab, F(ab′)₂, Fv, scFv, Fd, dAb, and other antibody fragments that retain antigen-binding function. Typically, such fragments would comprise an antigen-binding domain.

Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain includes an N-terminal variable (V) domain (VL) and a constant (C) domain (CL). Each heavy chain includes an N-terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3.

CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of immunological Interest, US Department of Health and Human Services (1991), eds. Kabat et al. Another standard for characterizing the antigen binding site is to refer to the hypervariable loops as described by Chothia. See, e.g., Chothia, D. et al.; J. Mol. Biol. 1992, Vol. 227: 799-817; and Tomlinson et al., EMBO J. 1995, Vol. 14: 4628-4638. Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops.

A Fab fragment (Fragment antigen-binding) consists of VH-CRI and VL-CL domains covalently linked by a disulfide bond between the constant regions. The Fᵥ fragment is smaller and consists of V_{H} and V_{L} domains non-covalently linked. To overcome the tendency of non-covalently linked domains to dissociate, a single chain Fᵥ fragment (scFᵥ) can be constructed. The scF, contains a flexible polypeptide that links (1) the C-terminus of V_{H} to the N-terminus of V_{L}, or (2) the C-terminus of V_{L} to the N-terminus of VH. A 15-mer (Gly₄Ser)₃ peptide may be used as a linker, but other linkers are known in the art.

The sequence of antibody genes after assembly and somatic mutation is highly varied, and these varied genes are estimated to encode 10¹⁰ different antibody molecules (Immunoglobulin Genes, 2nd ed., eds. Jonio et al., Academic Press 1995, San Diego, Calif.). The terms “antigen-binding domain” and “antigen-binding fragment” refer to a part of an antibody molecule that comprises amino acids responsible for the specific binding between antibody and antigen. The part of the antigen that is specifically recognized and bound by the antibody is referred to as the “epitope.” An antigen-binding domain may comprise an antibody light chain variable region (VL) and an antibody heavy chain variable region (VH); however, it does not have to comprise both. Fd fragments, for example, have two VH regions and often retain some antigen-binding function of the intact antigen-binding domain. Examples of antigen-binding fragments of an antibody include (1) a Fab fragment, a monovalent fragment having the VL, VH, CL and CH1 domains; (2) a F(ab′)₂ fragment, a bivalent fragment having two Fab fragments linked by a disulfide bridge at the hinge region; (3) a Fd fragment having the two VH and CH1 domains; (4) a Fv fragment having the VL and VH domains of a single arm of an antibody, (5) a dAb fragment (Ward et al., Nature 1989, Vola 341: 544-546), which has a VH domain; (6) an isolated complementarity determining region (CDR), and (7) a single chain Fv (scFv). Although the two domains of the Fv fragment, VL and VH are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., Science 1988, Vol. 242: 423-426; and Huston et al., Proc. Natl. Acad. Sci. USA 1988, Vol. 85: 5879-5883). These antibody fragments are obtained using conventional techniques known to those with skill in the art, and the fragments are evaluated for function in the same manner as are intact antibodies.

The term “human antibody” includes antibodies having variable and constant regions corresponding substantially to human germline immunoglobulin sequences known in the art, including, for example, those described by Kabat et al. (See Kabat, et al., Sequences of Proteins of Immunological Interest, Fifth Edition, U.S. Department of Health and Human Services, 1991 NIH Publication No. 91: 3242). The human antibodies applied in the methods and uses of the invention may include amino acid residues not encoded by human germline immunoglobulin sequences (e.g., mutations introduced by random or site-specific mutagenesis in vitro or by somatic mutation in vivo), for example in the CDRs, and in particular, CDR3. The human antibody can have at least one, two, three, four, five, or more positions replaced with an amino acid residue that is not encoded by the human germline immunoglobulin sequence.

Numerous methods known to those skilled in the art are available for obtaining antibodies or antigen-binding fragments thereof. For example, antibodies can be produced using recombinant DNA methods (U.S. Pat. No. 4,816,567). Monoclonal antibodies may also be produced by generation of hybridomas (see e.g., Kohler and Milstein, Nature 1975, Vol. 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; Smith, Science 1985, Vol. 228:1315-1317; Clackson et al., Nature 1991, Vol. 352: 624-628; Marks et al., J. Mol. Biol. 1991, Vol. 222: 581-597; WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al., Nature Genetics 1994, Vol. 7: 13-21, US 2003-0070185, WO 96/34096, published Oct. 31, 1996, and PCT Application No. PCT/US96/05928, filed Apr. 29, 1996.

In another embodiment, a monoclonal antibody is obtained from a non-human animal, and then modified, e.g., humanized, deimmunized, chimeric, may be produced using recombinant DNA techniques known in the art. A variety of approaches for making chimeric antibodies have been described. See e.g., Morrison era/., Proc. Natl. Acad. Sci. U.S.A. 81:6851, 1985; Takeda et al., Nature 1985, Vol. 314: 452, Cabilly et al., U.S. Pat. No. 4,816,567; Boss et al., U.S. Pat. No. 4,816,397; Tanaguchi et al., European Patent Publication EP 171 496; European Patent Publication 0 173 494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison, Science 1985, Vol. 229: 1202-1207; by Oi et al., BioTechniques 1986, Vol. 4: 214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

A humanized antibody can be optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A. 1983, Vol. 80: 7308-7312; Kozbor et al, Immunology Today 1983, Vol. 4: 7279; Olsson et al., Meth. Enzymol. 1982, Vol. 92: 3-16), and may be made according to the teachings of PCT Publication WO 92/06193 or EP 0 239 400).

An antibody or fragment thereof may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson et at., J. Mol. Biol. 1992, Vol. 227: 776-798; Cook, G. P. et al., Immunol. Today 1995, Vol. 16(5): 237-242; Chothia, D. et al., J. Mol. Biol. 1992, Vol. 227: 799-817; and Tomlinson et al., EMBO J. 1995, Vol. 14: 4628-4638. The V BASE directory provides a comprehensive directory of human immunoglobulin variable region sequences (compiled by Tomlinson, L A. Et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Likewise, an antibody applied in the kits, methods and uses of the invention can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, Annu. Rev. Immunol 1991, Vol. 9: 457-92; Capel et al., Immunomethods 1994, Vol. 4: 25-34 and de Haas et al., J. Lab. Clin. Med. 1995, Vol. 126: 330-41).

For additional antibody production techniques, see Antibodies: A Laboratory Manual, eds. Harlow et al., Cold Spring Harbor Laboratory, 1988. The antibody applied in the kits, methods and uses of the present invention is not necessarily limited to any particular source, method of production, or other special characteristics of an antibody.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 1990, Vol. 79: 315-321; Kostelny et al., J. Immunol. 1992, Vol. 148: 1547-1553. In one embodiment, the bispecific antibody comprises a first binding domain polypeptide, such as a Fab′ fragment, linked via an immunoglobulin constant region to a second binding domain polypeptide.

Small Modular Immunopharmaceuticals (SMIP™) provide an example of a variant molecule comprising a binding domain polypeptide. SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

A SMIP™ typically refers to a binding domain-immunoglobulin fusion protein that includes a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA₁ or the CH3 and CH4 regions of IgE (see e.g., U.S. 2005/0136049 by Ledbetter, J. et al., which is incorporated by reference, for a more complete description). The binding domain immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE).

The antibody applied in the kits, methods and uses of the present invention is an anti-CD31 antibody. In principle, each known or newly prepared anti-CD31 antibody is suitable. An anti-CD31 antibody is suitable if it has a high signal-to-background ratio when applied in the methods and uses described herein.

“Anti-CD31” means that the antibody is directed against CD31, binds to CD31 or reacts with CD31. Being directed to, binding to or reacting with includes that the antibody specifically binds to CD31. The term “specifically” in this context means that the antibody reacts with a CD31 protein but essentially not with another protein. For example, an anti-CD31 antibody directed against human CD31 does preferably not essentially bind to mouse CD31 protein. Likewise, an anti-CD31 antibody directed against mouse CD31 does preferably not essentially bind to human CD31. Accordingly, the term “another protein” includes any protein including proteins closely related to or being homologous to the CD31 protein against which the anti-CD31 antibody is directed to. For example, if the anti-CD31 antibody is directed to human CD31, it is preferred that it does not essentially bind to CD31 from another organism or species.

The term “does not essentially bind” means that the anti-CD31 antibody applied in the kits, uses and methods of the present invention does not bind another protein, i.e., shows a cross-reactivity of less than 30%, preferably 20%, more preferably 10%, particularly preferably less than 9, 8, 7, 6 or 5%.

Whether the antibody specifically reacts as defined herein above can easily be tested, inter alia, by comparing the reaction of said antibody with a CD31 protein with the reaction of said antibody with (an) other protein(s).

Accordingly, the anti-CD31 antibody applied in the kits, methods and uses of the invention preferably exerts a specific binding or specifically binds to CD31. The terms “specific binding” or “specifically binds” when used in the context of the anti-CD31 antibody applied in the kits, methods and uses of the invention refers to two molecules, i.e., CD31 expressed on a cell, preferably an endothelial cell involved in tumor vessel development in angiogenesis and an antibody binding to CD31, forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the binding affinity is of about 10⁻¹¹ to 10⁻⁸ M (KD), preferably of about 10⁻¹¹ to 10⁻⁹ M.

CD31 is a cluster of differentiation molecule. It is also called PECAM-1 for platelet endothelial cell adhesion molecule. Furthermore, it is also known as FLJ58394. Accordingly, the term “CD31”, “PECAM-1” and “FLJ58394” are used interchangeably in the present application. A human CD31 protein sequence is, for example, available in GenBank under Accession No. NP_{—}000433. CD31 from other species can be obtained by means known in the art.

CD31 plays a key role in removing aged neutrophils from the body. Macrophages palpate any passing neutrophil and have to decide whether the cell is healthy or has to be ingested.

In mature form, human CD31 has a 711 amino-acid sequence (polypeptide molecular weight: 79,578 daltons) which is depicted in SEQ ID NO:11. This sequence is merely an exemplary sequence, since the term “CD31” includes molecules from all mammalian species expressing CD31.

Usually, CD31 is glycosylated to the extent that approximately 39% of its molecular weight is attributable to carbohydrate, and the mature protein has nine putative, asparagine-linked glucosylation sites. All these sites are present in the glycoprotein extracellular domain, which has 574 amino acids. CD31 also includes a 19 amino-acid transmembrane portion and a 118 amino-acid cytoplasmic domain. This arrangement of domains is consistent with an orientation for PECAM-1 within the plasma membrane that is typical of other integral membrane glycoproteins.

In the context of the present invention, it has been found that CD31 is a particular useful marker for visualizing, i.e. in vivo imaging tumor vasculature and/or monitoring the therapeutic efficacy of an anti-angiogenic agent in a subject, since CD31 is a marker expressed during angiogenesis, particularly during angiogenesis as described herein.

The term “CD31” when used herein includes all forms (wild-type, splice variants, fragments, derivatives, analogs, proteins having an amino acid sequence which is at least 40, 50, 60, preferably 70, more preferably 80, even more preferred 90% identical to the amino acid sequence shown in SEQ ID NO:11) of CD31, with CD31 forms that are expressed on the surface of cells being preferred. Particularly preferred are CD31 proteins expressed by endothelial cells, preferably those endothelial cells involved in vessel development in angiogenesis, preferably in vessel development in tumor development. Cells expressing CD31 are, for example, mammalian cells or non-mammalian cells such as chicken or zebra fish cells, preferably mammalian cells such as mouse, rat, dog, cat, cow, chimpanzee or human cells, with human cells being particularly preferred.

Also exemplary of CD31 proteins within the present invention are molecules that correspond to a portion of CD31, or that comprise a portion of CD31 without being coincident with the natural molecule, and that display the adhesion-promoting activity of a CAM, as described below. Among these variants would be a polypeptide containing an amino-acid sequence that corresponds to the extracellular domain of CD31, absent the transmembrane or intracellular portions; that is, to a “soluble receptor” form of CD31.

Other CD31 proteins of the present invention are fragments of CD31 that retain CAM-like adhesion-promoting activity. Likewise within the present invention would be synthetic polypeptides that (i) correspond to a portion of the CD31 amino-acid sequence and (ii) retain an activity characteristic of CD31 as described below.

Whether a polypeptide is a CD31 protein of the invention can be routinely determined by means of an assay for a CD31 activity. Two such activities are the mediation, respectively, of adhesion-dependent movement of cells in response to a chemical attractant (chemotaxis) and of endothelial cell-cell adhesion. The former activity can be assayed, following Ohto, et al., Blood 1985, Vol. 66: 873-881, by determining whether an antibody that binds the synthetic polypeptide in question also inhibits chemotaxis of neutrophils or monocytes responding to a suitable stimulant, such as E. coli endotoxin. The ability to mediate adhesion between endothelial cells can be similarly assayed in routine fashion, i.e., by testing whether a polypeptide-recognizing antibody inhibits the anchoring of dispersed endothelial cells to a suitable substrate and/or the formation thereafter of intercellular junctions.

“Imaging” when used herein refers to any technique and process used to create images of the human body (or parts thereof), preferably for clinical purposes (medical procedures seeking to reveal, diagnose or examine disease) or medical science (including the study of normal anatomy and physiology). Preferably, imaging when applied in the methods and uses of the invention is used to create images of the tumor vasculature of a subject.

The imaging is done in the context of the present invention “in vivo”. “In vivo imaging” refers to localizing a ligand of interest using an imaging or scanning technology in a living subject. Accordingly, the methods and uses of the present invention which apply in vivo imaging are not carried out in a partial or dead organism.

In the context of the present invention the ligand of interest is CD31 which is localized, i.e. bound by a fluorescence labelled anti-CD31 antibody that is detectable by any suitable imaging or scanning technology known in the art, with near infra-red fluorescent imaging being preferred.

The methods and uses of the invention are non-invasive. “Non-invasive” means that no break in the skin of a subject is created, for example, an incision, and there is no contact with the mucosa, or skin break, or internal body cavity beyond a natural or artificial body orifice. For example, imaging through the ear-drum or inside the nose or a wound dressing change all fall outside the definition of “non-invasive”.

“Vasculature” when used herein refers to the arrangement or the distribution of blood vessels in an organ or body part (e.g. the vasculature of the brain, of the lungs, of the kidneys, or of the heart itself). The vasculature is the network of blood vessels of an organ or body part; it includes all vessels of the body from the aorta to arteries, capillaries and veins.

A vasculature can only form if blood vessels have been formed by angiogenesis. As explained herein, a tumor can initiate angiogenesis, thereby blood vessels are formed. These blood vessels then constitute the vasculature. Accordingly, when used herein the term “vasculature” encompasses the process of angiogenesis and thus the formation of blood vessels, for example, tumor blood vessels.

The term “tumor vasculature” refers to the vasculature of tumor, preferably of a tumor as described herein. Accordingly, as explained elsewhere herein a subject when used herein has preferably a tumor.

A blood vessel is a vessel lined with endothelium through which blood circulates and which is built by an endothelial cell, i.e., a vasculature cell. The circulatory system used to transport around the body consists of many tubes that control the flow of blood around the body, these are blood vessels, consisting primarily of arteries and veins.

Tumour blood vessels have perivascular detachment, vessel dilation, and irregular shape. It is believed that tumor blood vessels are not smooth like normal tissues and are not ordered sufficiently to give oxygen to all of the tissues. Endothelial precursor cells are organized from bone marrow, which are then integrated into the growing blood vessels. Then the endothelial cells differentiate and migrate into perivascular space, to form tumour cells. Vascular endothelial growth factor (VEGF) plays a crucial role in the formation of blood vessels that lead to tumor growth, which allows the vessel to expand. It is called sprouting angiogenesis.

Accordingly, the present invention also contemplates methods and uses of to in vivo imaging, i.e., monitoring angiogenesis during tumor development by detecting a fluorescence labelled anti-CD31 antibody. CD31 being a particularly suitable angiogenesis marker.

As mentioned herein, CD31 is a particular useful marker for visualizing tumor vasculature, since CD31 is a marker expressed during angiogenesis, thereby resulting in the formation of blood vessels.

“Angiogenesis” is the formation of thin-walled endothelium-lined structures with/without muscular smooth muscle wall and pericytes (fibrocytes).

When used herein said term also refers to physiological processes involving the growth of new blood vessels from pre-existing vessels. In the context of the present invention by the term “angiogenesis” preferably angiogenesis during tumor development is meant. Accordingly, said term does not only refer to the process of angiogenesis, but also to the process of vasculogenesis and/or arteriogenesis.

“Vasculogenesis” is the formation of vascular structures from circulating or tissue-resident endothelial stem cells (angioblasts), which proliferate into de novo endothelial cells. This form particularly relates to the embryonal development of the vascular system.

“Arteriogenesis” is the formation of medium-sized blood vessels possessing tunica media plus adventitia

Angiogenesis is a normal process in growth and development, as well as in wound healing. Angiogenesis is stimulated by various growth factors such as vascular endothelial growth factor (VEGF) and fibroblast growth factor (FGF). An early response to angiogenesis-stimulating factors is the degradation of the endothelial cell basement membrane by proteases such as members of the matrix metalloproteinase (MMP) family. MMPs degrade collagen and other extracellular matrix components. Destroying the basement membrane barrier, allows endothelial cells to migrate from pre-existing vessels towards angiogenic stimuli and to proliferate (Marsh, Stem Cells 1997; Vol. 15: 180-189). Vascular cell-adhesion molecules contribute to endothelial cell migration by mediating cell-extracellular-matrix interactions. One of the major mediators is the integrin αᵥβ₃, a receptor for proteins such as fibronectin. Antagonists of integrin αᵥβ₃ inhibit the growth of new blood vessels, suggesting that cell adhesion is a critical step in angiogenesis (Brooks et al., Cell 1994; Vol. 79: 1157-1164). Following migration and proliferation, endothelial cells assemble into tubes with a patent lumen. An important step in producing a mature blood vessel is the recruitment of mesenchymal cells and their subsequent differentiation into smooth muscle cell-like pericytes that are thought to stabilize the newly forming vasculature. These pericytes are gradually attracted by an endothelial plexus leading to progressive covering of the vascular tree (Benjamin et al., Development 1998; Vol. 125:1591-1598). During this period, regression of uncovered capillaries occurs, which ceases after acquisition of a pericyte coating. This later phase of blood vessel formation, therefore, is characterized by remodelling steps in which unprotected vessels are pruned and pericyte-covered vessels become stabilized and less prone to destabilization and regression. Platelet derived growth factor (PDGF) probably derived from an endothelial cell source, recruits pericytes to associate with endothelial cells to increase blood vessel stability (Lindahl et al., Science 1997; Vol. 277: 242-245). The afore-described processes in angiogenesis are also illustrated in FIG. 1.

In a second aspect the present invention relates to a non-invasive method of in vivo monitoring the therapeutic efficacy of an anti-angiogenic agent in a subject comprising detecting a fluorescence labelled anti-CD31 antibody.

The term “in vivo monitoring the therapeutic efficacy of an anti-angiogenic agent” also includes monitoring the therapeutic efficacy of an anti-angiogenic agent on angiogenesis, preferably angiogenesis during tumor development.

Accordingly, the present invention furthermore envisages methods and uses to in vivo image, i.e., monitor the therapeutic effect of an anti-angiogenic agent on angiogenesis by detecting a fluorescence labelled anti-CD31 antibody. CD31 being a particularly suitable angiogenesis marker.

The embodiments described in the context of the non-invasive method of in vivo imaging tumor vasculature in a subject by detecting a fluorescence labelled anti-CD31 antibody also apply to the second aspect of the present invention.

The term “monitoring” when used in the context of the methods and uses of the invention includes that blood vessels, in particular blood vessels and/or vasculature, in particular tumor vasculature, is in vivo visualized by appropriate means and methods. It also includes that an increase or decrease of blood vessels of the vasculature, preferably tumor vasculature, can be visualized by the signals from the fluorescence labelled anti-CD31 antibody signal over time due to its binding (or non-binding) to CD31 on the surface of a vasculature cell, i.e., the less blood vessels, the less is the signal provided by the antibody or, likewise, the more blood vessels, the more is the signal provided by the antibody. It also includes that angiogenesis, preferably angiogenesis during tumor development is monitored and/or that the therapeutic efficacy of an anti-angiogenic effect on tumor vasculature and/or angiogenesis is monitored. It furthermore includes that the size of a tumor and/or tumor blood vessels is monitored and/or measure.

As used herein, “therapeutic efficacy” of an anti-angiogenic agent refers to the degree of efficacy of an anti-angiogenic agent upon single or multiple dose administration to a subject (such as a human patient) at reducing or preventing angiogenesis, preferably angiogenesis in tumor development. Reducing or preventing angiogenesis in tumor development is a measure in tumor treatment.

The therapeutic effect which an anti-angiogenic agent may be detected by all established methods and approaches which will indicate a therapeutic effect. It is, for example, envisaged that the therapeutic effect is detected by way of surgical resection or biopsy of an affected tissue/organ which is subsequently analyzed by way of immunohistochemical (IHC) or comparable immunological techniques. Alternatively, as an indirect measurement of the therapeutic efficacy of an anti-angiogenic agent, it is also envisaged that the tumor markers in the serum of the patient (if present) are detected in order to diagnose whether the therapeutic approach is already effective or not. Additionally or alternatively it is also possible to evaluate the general appearance of the respective patient (fitness, well-being, decrease of tumor-mediated ailment etc.) which will also aid the skilled practitioner to evaluate whether a therapeutic effect is already there. The skilled person is aware of numerous other ways which will enable him or her to observe a therapeutic effect. For cancer therapy, therapeutic efficacy can be measured, for example, by assessing the time to disease progression (TTP) and/or determining the response rate (RR). Specifically, an anti-angiogenic agent may cause a reduction or, more preferably elimination of the tumor. Thus, the therapeutic efficacy of an anti-angiogenic agent may be measured/judged on the basis of TTP and/or the RR.

The term “treatment” refers to a therapeutic or preventative measure. The treatment may be administered to a subject having a tumor such as a solid tumor or who ultimately may develop a tumor, in order to prevent, reduce and/or cure tumor development or in order to prolong the survival of a subject beyond that expected in the absence of such treatment. For the purpose of the present invention a subject having a tumor suffers from cancer. Accordingly, the methods and uses of the invention are applied to a subject suffering from cancer.

In some embodiments the subject has a solid tumor. A solid tumor is an abnormal mass of tissue that usually does not contain cysts or liquid areas. Solid tumors may be benign (not cancer), or malignant (cancer).

For example, a solid tumor refers to a tumor selected from the group of gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

For the purpose of the present invention, a solid tumor has a size of at least 1 mm, preferably 2 mm. At this size, a solid tumor in order to proliferate and to survive depends on an adequate supply of growth factors and the removal of toxic molecules as well as on a sufficient oxygen supply. In solid tissues, oxygen, for example, can diffuse radially from capillaries for only about 150 to 200 μm and, thus, the tumor depends on the supply of oxygen by blood vessels and, thus, initiates angiogenesis as described herein.

Expansion of tumor masses beyond about 1 or 2 mm in diameter depends on the development of an adequate blood supply. Accordingly, the tumor starts to supply itself with blood and, therefore, the phenomenon of tumor angiogenesis occurs. Thanks to the methods and uses of the present invention it is possible to image tumor vasculature and/or monitor the therapeutic efficacy of an anti-angiogenic agent in a subject, thereby providing support in the treatment of cancer.

The methods and uses of the present invention for in vivo imaging tumor vasculature or in vivo monitoring the therapeutic efficacy of an anti-angiogenic agent in a subject also allow monitoring the size of a tumor. Hence, conclusions can be drawn during and/or after the treatment of a subject with an anti-angiogenic agent as to whether such an agent may reduce the size of a tumor. Accordingly, when the methods and uses of the present invention are applied for in vivo imaging tumor vasculature or in vivo monitoring the therapeutic efficacy of an anti-angiogenic agent in a subject, they may optionally also be applied to monitor and/or measure the size of a tumor. Thus, the terms “in vivo imaging tumor vasculature” and “in vivo imaging the therapeutic effect of an anti-angiogenic agent” also include monitoring and/or measuring the size of a tumor.

The size of the tumor when measured by conventional imaging techniques such as computer tomography suffer from the disadvantage that no conclusion can be drawn as to whether the tumor is still “active”, i.e., whether it is metabolically active and alive. For example, a tumor may have a large size after, for example, having been treated with an anti-angiogenic agent and thus, it may be concluded that the treatment was not successful. However, in reality the treatment was successful, since the tumor, albeit being still large, is no longer active in angiogenesis. Thus, one would come to a false-positive result. Likewise, one could come to a false-negative result, if it is observed that the tumor is small in size, while being active in angiogenesis.

However, thanks to the present invention, the methods and uses described herein do not only allow monitoring and/or measuring the size of a tumor, they also allow to measure and/or monitor the angiogenic activity of the tumor. Thus, it allows making a correlation between tumor size and angiogenic activity. Specifically, the fluorescence labelled anti-CD31 antibody is stable enough so that tumors can be explanted and/or biopsies can be taken which may then be analysed in vitro, for example, as described herein above. More specifically, one can determine the signal of the anti-CD31 antibody and, thus, could conclude as to whether the treatment the tumor was successful, albeit the tumor is still large in size and vice versa.

The term “cancer” refers to or describes the physiological condition in a subject that is typically characterized by unregulated cell growth. Examples of cancer or tumors include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

As used herein, the term “anti-angiogenic agent” refers to a molecule that reduces or prevents angiogenesis, which is responsible for the growth and development of blood vessels.

An anti-angiogenic agent in accordance with the present invention may therefore stop or inhibit the growth of primary tumors, impede or reduce the formation of metastases, and impede the appearance of secondary growths. Angiogenic inhibitors are also useful in the treatment of non-neoplastic disorders in which an angiogenic activity occurs.

The anti-angiogenic agent is, for example, an inhibitor of VEGF-A, such as the antibody sold under the tradename Avastin®, VEGF-B, VEGF-C, VEGF-D, VEGFR1, VEGFR2, or Angiopoietin. The inhibitor is, for example, an antibody, siRNA or a small molecule. Furthermore, a variety of anti-angiogenic agents is useful in the invention and can be prepared by routine methods. Such anti-angiogenic agents include, without limitation, small molecules; proteins such as dominant negative forms of angiogenic factors, transcription factors and antibodies; peptides and peptidomimetics; and nucleic acid molecules including ribozymes, antisense oligonucleotides, and nucleic acid molecules encoding, for example, dominant negative forms of angiogenic factors and receptors, transcription factors, and antibodies and antigen-binding fragments thereof. See, for example, Hagedorn and Bikfalvi, Crit. Rev. Oncol. Hematol. 2000, Vol. 34: 89-110, and Kirsch et al., J. Neurooncol. 2000, Vol. 50: 149-163.

The anti-angiogenic effect of an anti-angiogenic agent may be tested by using a number of in vitro and in vivo bioassays such as the proliferation assay and the chemotaxis assay. The proliferation assay uses cultured capillary endothelial cells and measures either increased cell numbers or the incorporation of radiolabelled or modified nucleosides to detect cells in S-phase. In contrast, the chemotaxis assay separates endothelial cells and a test solution by a porous membrane disc, such that migration of endothelial cells across the barrier is indicative of a chemoattractant present in the test solution (Hanahan and Folkman, Cell 1996; Vol. 86: 353-364). By these assays also further anti-angiogenic agents may be identified.

Vascular endothelial growth factor (VEGF) has been shown to be important for angiogenesis in many types of cancer, including breast cancer angiogenesis in vivo (Borgstrom et al., Anticancer Res. 1999, Vol. 19: 4213-4214). The biological effects of VEGF include stimulation of endothelial cell proliferation, survival, migration and tube formation, and regulation of vascular permeability. An anti-angiogenic agent useful in the invention can be, for example, an inhibitor or neutralizing antibody that reduces the expression or signalling of VEGF or another angiogenic factor, for example, an anti-VEGF neutralizing monoclonal antibody (Borgstrom et al., supra, 1999). An anti-angiogenic agent also can inhibit another angiogenic factor such as a member of the fibroblast growth factor family such as FGF-1 (acidic), FGF-2 (basic), FGF-4 or FGF-5 (Slavin et al., Cell Biol. Int. 1995, Vol. 19: 431-444; Folkman and Shing, J. Biol. Chem. 1992, Vol. 267: 10931-10934) or an angiogenic factor such as angiopoietin-1, a factor that signals through the endothelial cell-specific protein Tie2.

A variety of other molecules can also function as anti-angiogenic agents useful in the invention including, without limitation, angiostatin; a kringle peptide of angiostatin; endostatin; anastellin, heparin-binding fragments of fibronectin; modified forms of antithrombin; collagenase inhibitors; basement membrane turnover inhibitors; angiostatic steroids; platelet factor 4 and fragments and peptides thereof; thrombospondin and fragments and peptides thereof; and doxorubicin (O'Reilly et al., Cell 1994, Vol. 79: 315-328); O'Reilly et al., Cell 1997, Vol. 88: 277-285; Homandberg et al., Am. J. Path. 1985, Vol. 120: 327-332; Homandberg et al., Biochim. Biophys. Acta 1986, Vol. 874: 61-71; and O'Reilly et al., Science 1999, Vol. 285: 1926-1928). Commercially available anti-angiogenic agents useful in the invention include, for example, angiostatin, endostatin, metastatin and 2ME2 (EntreMed; Rockville, Md.); anti-VEGF antibodies such as Avastin (Genentech; South San Francisco, Calif.); and VEGFR-2 inhibitors such as SU5416, a small molecule inhibitor of VEGFR-2 (SUGEN; South San Francisco, Calif.) and SU6668 (SUGEN), a small molecule inhibitor of VEGFR-2, platelet derived growth factor and fibroblast growth factor I receptor. It is understood that these and other anti-angiogenic agents can be prepared by routine methods and are encompassed by the term “anti-angiogenic agent” as used herein.

In a preferred aspect of the methods and uses of the invention the solid tumor of the subject is a xenograft, preferably a human xenograft.

This aspect contemplates a pre-clinical model for testing an anti-angiogenic effect of a compound, i.e. a (potential) anti-angiogenic agent. Accordingly, the subject having the solid tumor xenograft, preferably a solid tumor from human, is a non-human mammal, preferably a rodent such as a rabbit, rat and mouse, with mouse being preferred, or a chimpanzee. The anti-CD31 antibody applied in this aspect is an anti-CD31 antibody directed against mouse CD31 which does not essentially bind another protein such as human CD31. Most preferably, this antibody can be obtained from Acris Antibodies, 32052 Herford, Germany (clone 2H8, catalogue No. SM007A). It is a hamster anti-mouse CD31 antibody. This antibody is also described in Bogen et al., Am. J. Pathol 1992, Vol. 141: 843-854; Bogen et al., J. Exp. Med. 1994, Vol. 179: 1059-1064 and Ishikaw et al., Jpn J. Pharmacol. 2002, Vol. 88: 332-340.

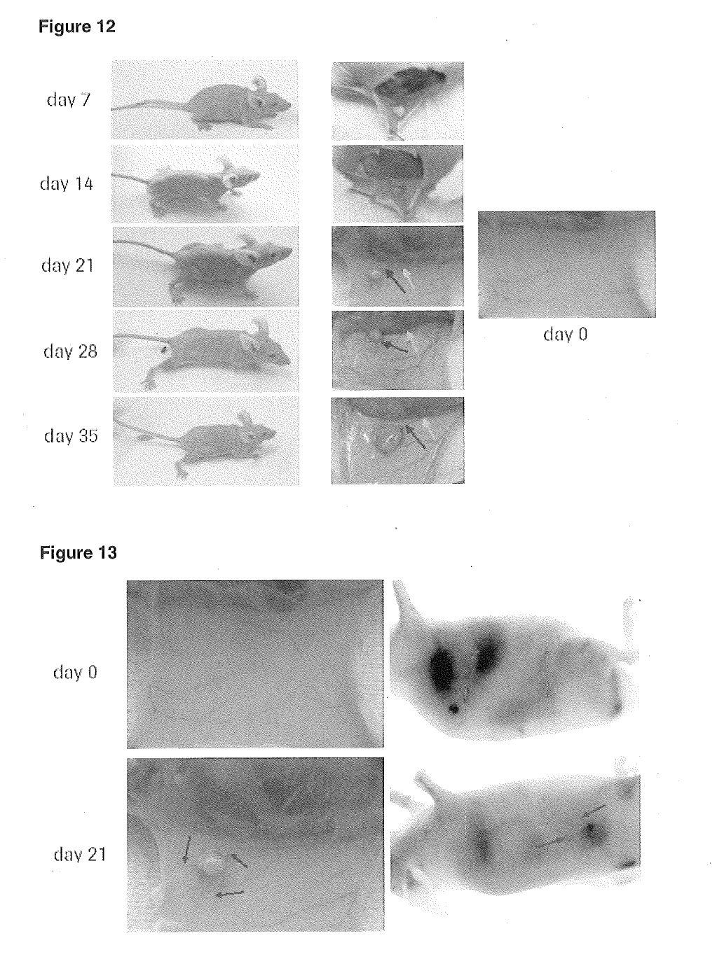

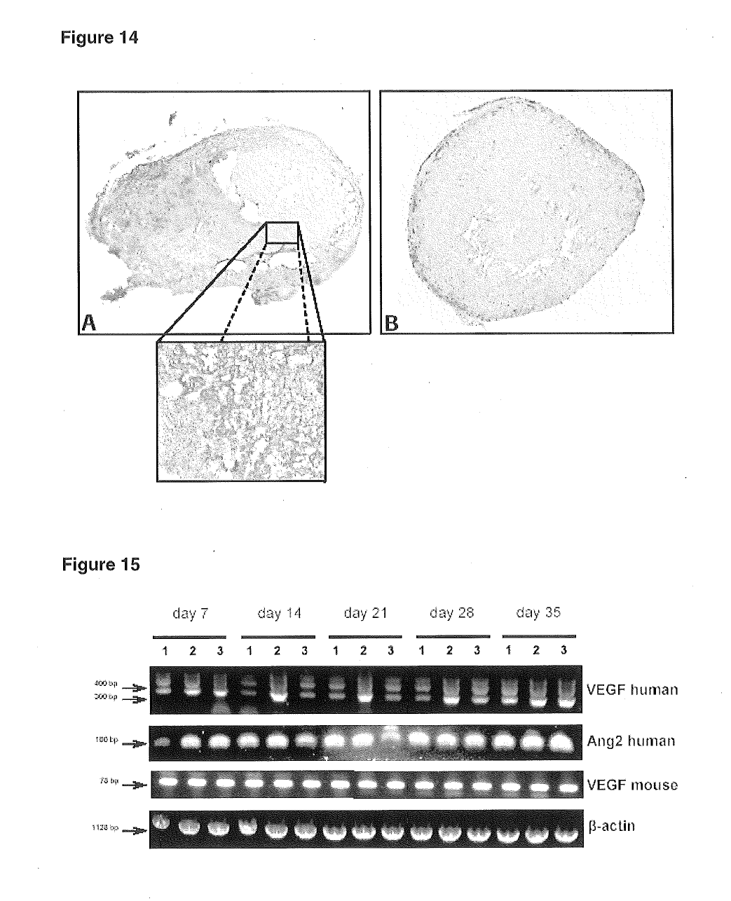

In particular, the inventors have observed that after subcutaneous cell inoculation of Calu3 cells into mice, tumors revealed an initial uniform expansion. At day 7, the first day of tumor size measurement, the average tumor volume was higher than the following ones (see FIG. 11). Cells first need to arrange themselves, in a coordinated fashion manner to establish a solid tumor. At study day 35, the start of therapy, the tumors reached an average tumor volume of approximately 150 mm³. Until this time point and apart from the first measurement, tumors exhibited an exponential growth with a tumor growth rate of about 40-50 mm³ per week. At day 7, tumors showed no obvious vascularization. They did not reveal the typical color of tumors indicating that functional blood supply had not been established. Newly formed blood vessel could not be detected around the tumors (see FIG. 12). At day 14, in the area surrounding the subcutaneously inoculated human tumor cells, an approx. 1 mm red zone around the tumor appeared. This zone consisted of engorged small capillaries that formed a dense network of many interlacing vessels. At day 21, the vascular engorgement extended into large vessels (see FIG. 12). Imaging results obtained with Qtracker705 non-targeted quantum dots demonstrated the vascular changes in the mouse tissue and the growth of vessels around and into the tumor tissue (see FIG. 13). At study day 35, the number of tumor blood vessels increased and thus, tumors revealed a reddish color suggesting a well established blood supply within the tumors. Immunohistochemical evaluation using an alkaline phosphatase system followed by the visualization of mouse endothelial CD31+ cells using a slide scanner, allowed a better visualization of blood vessel distribution within the whole tumor. Cryoslides at day 7, demonstrated how mouse tissue is incorporated into the human tumors (see FIG. 14 A). Murine stroma cells were migrating into the human cancer tissue connecting first blood vessels to the tumors. The blood vessel distribution pattern of the established human tumors at day 21, revealed an accumulation of blood vessels at the tumor outlines (see FIG. 14 B). Major arteries gathered at the tumor edges whereas the inner part of the tumor was permeated with small and engorged capillaries. mRNA expression profiling during the first 35 days of tumor development revealed no dramatic changes in the gene expression of angiogenic growth factors. At day 7, 14, 21, 28 and 35, respectively 3 tumors were explanted and prepared for mRNA extraction as described herein. Detection of 13-actin was included as a loading control. VEGF (human), exhibited two major splice variants involved in angiogenic development. Those splice variants alternated in different states of tumor development. At day 7, generally, levels of splice variant 5 (462 bp) were higher than in later phases and expression of splice variant 6 (365 bp) was lower in early states of tumor development (see FIG. 15). The levels of Ang2 (human) were increased progressively from day 7 up to day 35, whereas VEGF (mouse) levels were not changed (see FIG. 15). Ang2 (mouse) was completely absence (data not shown) or inappropriate RT-PCR condition were used. The presence of angiogenic growth factors are a rationale for the application of anti-angiogenic therapies with target-specific therapeutic antibodies.

Immunohistochemical evaluation using an alkaline phosphatase system followed by the visualization of mouse endothelial CD31+ cells using a slide scanner, allowed a better visualization of blood vessel distribution within the whole tumor. Cryoslides at day 7, demonstrated how mouse tissue is incorporated into the human tumors (see FIG. 14 A). Murine stroma cells were migrating into the human cancer tissue connecting first blood vessels to the tumors. The blood vessel distribution pattern of the established human tumors at day 21, revealed an accumulation of blood vessels at the tumor outlines (see FIG. 14 B). Major arteries gathered at the tumor edges whereas the inner part of the tumor was permeated with small and engorged capillaries.

mRNA expression profiling during the first 35 days of tumor development revealed no dramatic changes in the gene expression of angiogenic growth factors. At day 7, 14, 21, 28 and 35, respectively 3 tumors were explanted and prepared for mRNA extraction. Detection of β-actin was included as a loading control. VEGF (human) exhibited two major splice variants involved in angiogenic development. Those splice variants alternated in different states of tumor development. At day 7, generally, levels of splice variant 5 (462 bp) were higher than in later phases and expression of splice variant 6 (365 bp) was lower in early states of tumor development (see FIG. 15). The levels of Ang2 (human) were increased progressively from day 7 up to day 35, whereas VEGF (mouse) levels were not changed (see FIG. 15). Ang2 (mouse) was completely absence (data not shown) or inappropriate RT-PCR condition were used. The presence of angiogenic growth factors are a rationale for the application of anti-angiogenic therapies with target-specific therapeutic antibodies.

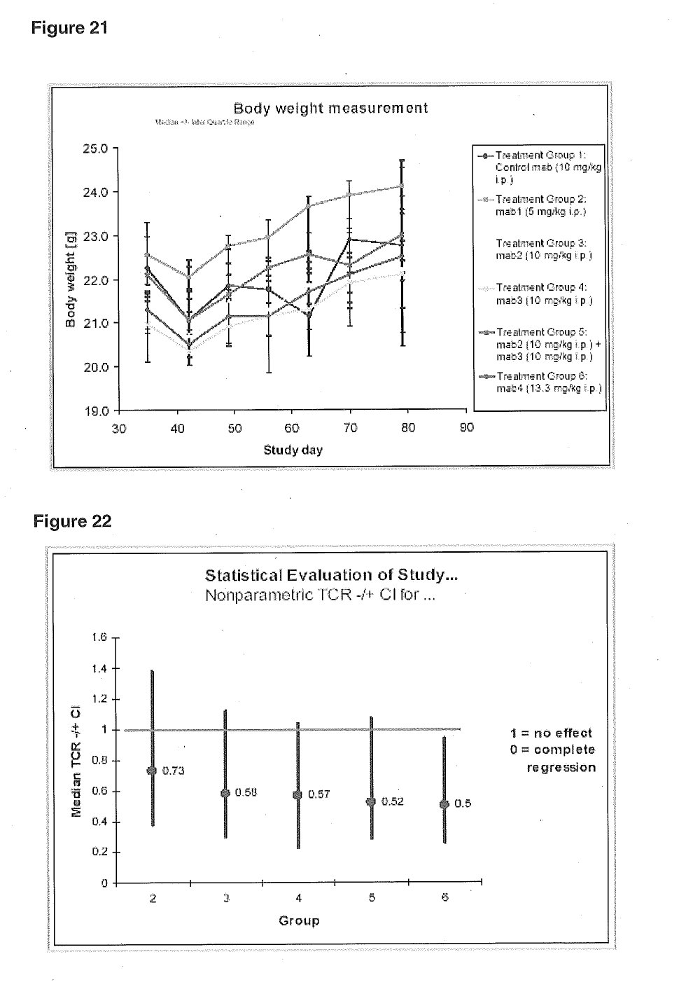

At study day 35, mice were randomized to statistically well distributed groups, depending on their body weight and tumor size. For the treatment with therapeutic antibodies, each group consisted of 10 Balbc/nude mice bearing Calu3 tumors and treatment with therapeutic antibodies was applied for a 6 week time period. Tumor size and body weight measurement were carried out once a week. All therapeutic groups revealed an average tumor growth inhibition in a range of 27-50% relative to the control mab (see FIG. 20). Body weight measurement was performed to monitor the general conditions of mice. No abnormalities were observed (see FIG. 21). Both measurements were analyzed nonparametric (median) because uniform distribution within one group was not observed.

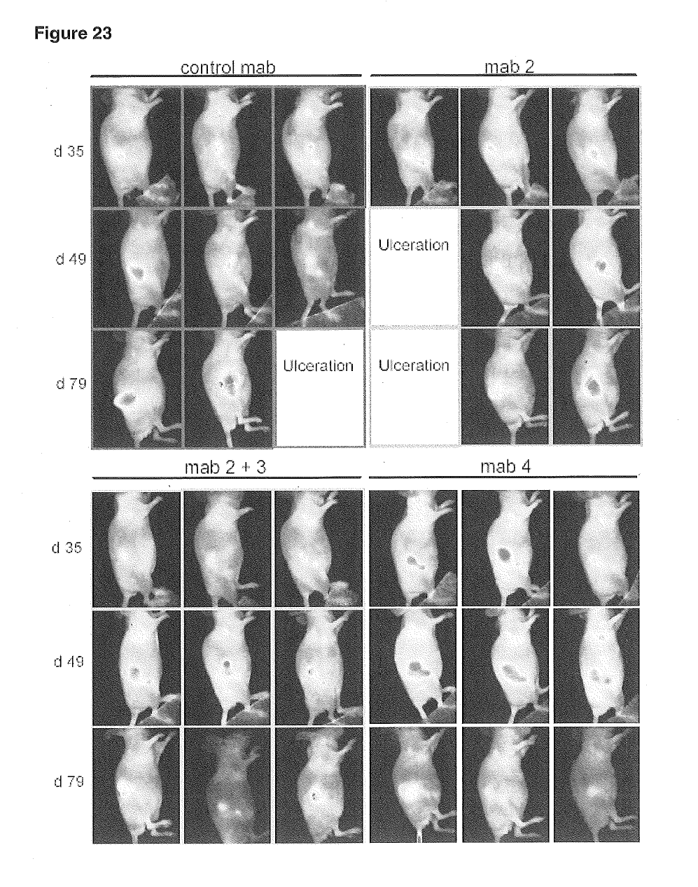

The treatment-to-control ratio (TCR) and its two-sided non-parametric confidence interval (CI) were determined using the SAS program TUMGRO and the AUC evaluation. This nonparametric analysis revealed that by using a level of significance of 5% (á), only the treatment group 6 with mab 4 is statistically significant relatively to control mab. Treatment groups 2-4 were not statistically significant (see FIG. 22).

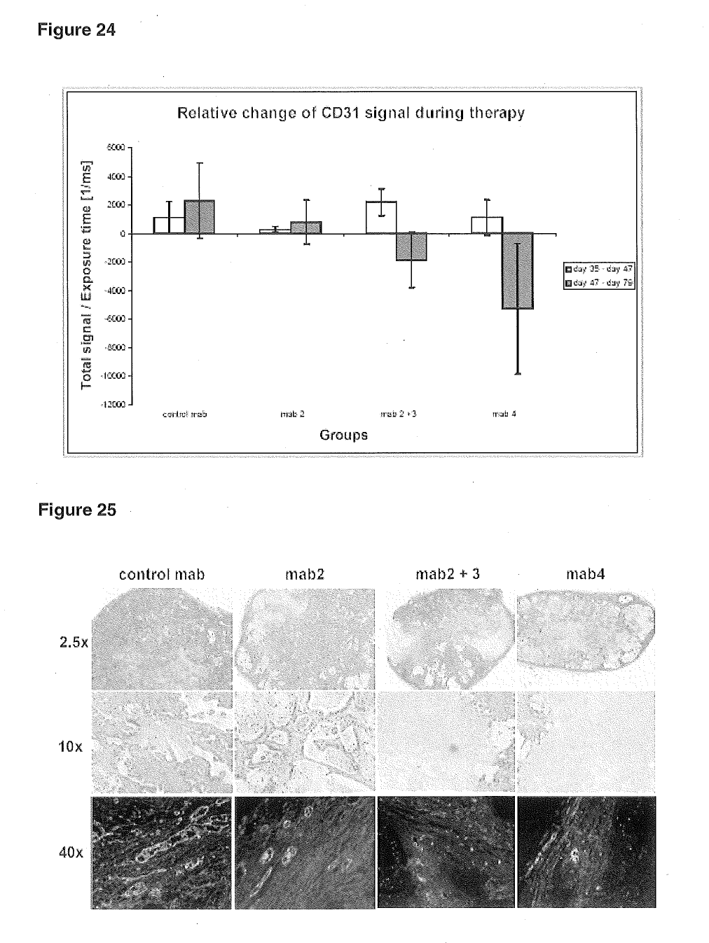

Blood vessel monitoring with anti-CD31 Preliminary studies revealed that anti-CD31 antibody was the best agent for imaging tumor vasculature. This agent is targeting mouse endothelial CD31 receptors and visualizes single blood vessels with a high signal-to-background ratio. Therefore, imaging for anti-CD31 antibody represents a feasible way to image tumor vasculature. Three mice of each therapy group have been chosen and injected i.v. with 50 μg/mouse anti-CD31 antibodies at day 35, 49 and 79. Near-infrared imaging was carried out 24 hrs post-reagent-injection after anesthetizing the animals. During the study, some mice had to be excluded because they revealed tumor ulcerations that falsify the imaging results. An increase or decrease of tumor vasculature was visualized by using the compare image tool of the MAESTRO system (see FIG. 23). Control mab and mab 1 treatment groups showed an obvious increase of tumor blood vessels from day 35 to day 79, whereas the combined treatment with mab 2+3 and mab 4 exhibited a decrease of tumor vasculature. Tumor regions were quantified by manually drawing measurement areas and signal intensities were evaluated in intensity values (total signal/exposure time). The average changes of CD31 signals from day 35 to 49 and from day 49 to 79 were plotted in chart 28. All treatment groups revealed an increase in tumor vasculature from day 35 to 49. While CD31 tumor signals steadily accelerated in group 1 (control mab) and group 3 (mab 2), tumor vasculature significantly decreased in group 5 and 6 (see FIG. 24).

Immediately after the last in vivo imaging studies, tumors were explanted (day 79), fixed in formalin and embedded in paraffin for ex vivo studies. Fluorescence microscopy showed numerous well defined capillaries in tumors treated with control mab. Several tumor blood vessels were observed in mice treated with mab 2. In contrast, treatment groups 5 and 6 had obviously fewer and less defined blood vessels in the tumors compared to other treatment groups. They revealed lower microvessel density, capillaries were generally smaller and unstructured and they exhibited weaker anti-CD31 fluorescence signals. Histochemical HE-staining showed intratumoral necrotic regions for up to 90% in the treatment group with mab 4. Those areas of tumor necrosis were significantly higher in group 5 and 6 than in the other treatment groups. In contrast, tumors in the control group were distributed continuously with intact tumor cells (see FIG. 25).

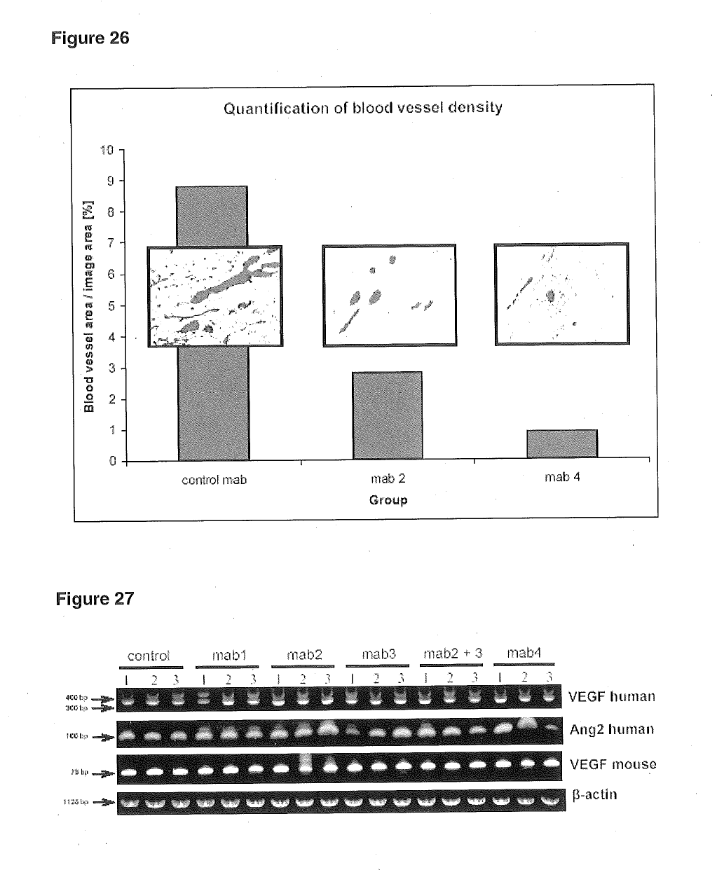

Blood vessel density in paraffin sections was determined by a custom written MATLAB script. Blood vessel regions were with 8.8% significantly larger in the control treatment group than in the groups treated with therapeutic antibodies. 2.8% of capillary areas were observed in mab 2 treatment group, whereas only 0.9% blood vessel presence was calculated for group treated with mab 4 (see FIG. 26).

The mRNA expression profile of angiogenic growth factors during therapy did not reveal any changes in the expression pattern. At day 79, respectively 3 tumors were explanted and prepared for mRNA extraction. Detection of β-actin was included as a loading control. As mentioned, VEGF (human), exhibited two major splice variants, but at this point the alternation did not appear. The level of VEGF (human) was comparable in all therapeutic groups as well as the Ang2 (human) and VEGF (mouse) levels (see FIG. 27). Ang2 (mouse) was completely absence (data not shown) or inappropriate RT-PCR condition were used.

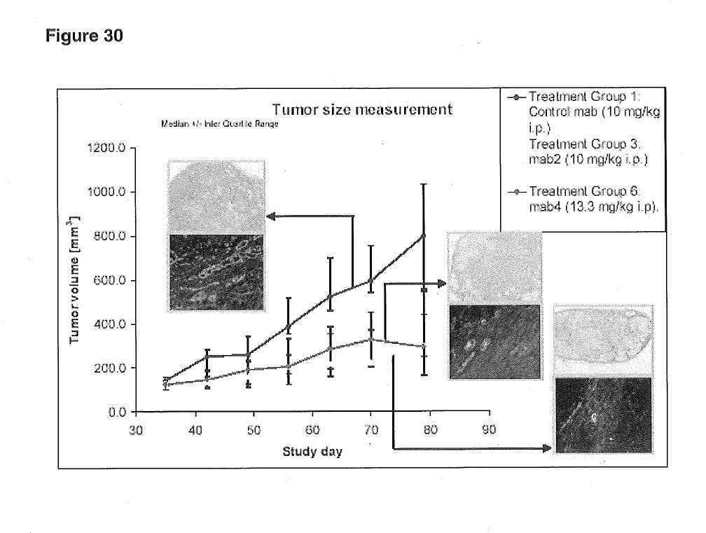

All treatment groups reveal tumor inhibitions up to 50% relative to the control group. However, statistically significant tumor inhibition is only observed in treatment group with Tumor volumes for group mab 2 and group mab 4 exhibit a similar growth curve development. It could be assumed that vascularization patterns are similar and thus, reveal similar anti-CD31 signals. However, blood vessel monitoring with anti-CD31 shows an increase of tumor vasculature in group mab 2 and a decrease of the anti-CD31 signal in group with mab 4 (see FIG. 24). Furthermore, tumors treated with mab 4 reveal up to 95% necrotic regions suggesting a functional destruction or inhibition of proper blood vessel development. Treatment group mab 2 exhibit roughly 70% necrotic regions and a blood vessel density that is almost three times higher than in group mab 4 (see FIG. 26). These results suggest that there is no correlation between tumor vasculature and tumor volume. It shows that mab 4 and mab 2 differ in there ability to inhibit tumor blood vessel formation at the molecular level although, tumor volumes do not differ significantly. The assumption that mRNA expressions of angiogenic growth factors could be modulated during the treatment with target specific antibodies, could not be confirmed. The mRNA expression of VEGF and Ang2 is neither up-regulation nor down-regulated in the different treatment groups. However, for more accurate analysis, mRNA expression profiling should be carried out using a light-cycler system that enables a more quantitative evaluation. The different tumor inhibition effects of the therapeutic antibodies could be explained by the dosage of mab applied. Mab 1 for example, was injected at a concentration of 5 mg/kg and showed minimal tumor inhibition. In contrast, Mab 4 with 13.3 mg/kg, exerted the highest tumor inhibition. However, dosages have been selected based on preliminary efficacy studies. Mab 1, in higher concentrations, causes serious side effects and therefore preclinical studies had to be terminated. Further studies for the comparison of antibody based angiogenic effects should be performed with equal antibody concentrations. Mab 4. Blood vessel formation monitoring with anti-CD31 antibody at different time points demonstrates an increase of tumor vasculature in the control treatment group, whereas treatment groups with Mab 2+3 and Mab 4 shows a prominent decrease of tumor vasculature during therapy. Histological HE-staining of explanted tumor tissue reveals highly necrotic regions in tumors treated with Mab 2+3 and Mab 4 (see FIG. 30). The blood vessel density in these groups is much smaller than in those treated with the control Mab (see FIG. 26).

Tumor volumes for group Mab 2 and group Mab 4 exhibit a similar growth curve development. It could be assumed that vascularization patterns are similar and thus, reveal similar anti-CD31 signals. However, blood vessel monitoring with anti-CD31 shows an increase of tumor vasculature in group Mab 2 and a decrease of the anti-CD31 signal in group with Mab 4 (see FIG. 24). Furthermore, tumors treated with Mab 4 reveal up to 95% necrotic regions suggesting a functional destruction or inhibition of proper blood vessel development. Treatment group Mab 2 exhibit roughly 70% necrotic regions and a blood vessel density that is almost three times higher than in group Mab 4 (see FIG. 26). These results suggest that there is no correlation between tumor vasculature and tumor volume. It shows that Mab 4 and Mab 2 differ in there ability to inhibit tumor blood vessel formation at the molecular level although, tumor volumes do not differ significantly. The assumption that mRNA expressions of angiogenic growth factors could be modulated during the treatment with target specific antibodies could not be confirmed. The mRNA expression of VEGF and Ang2 is neither up-regulation nor down-regulated in the different treatment groups. However, for more accurate analysis, mRNA expression profiling should be carried out using a light-cycler system that enables a more quantitative evaluation. The different tumor inhibition effects of the therapeutic antibodies could be explained by the dosage of mab applied. Mab 1 for example, was injected at a concentration of 5 mg/kg and showed minimal tumor inhibition. In contrast, Mab 4 with 13.3 mg/kg, exerted the highest tumor inhibition.

The above presented results demonstrate that a rodent, preferably a mouse xenograft tumor model, is suitable for determining as to whether an agent may have an anti-angiogenic effect which can be visualized by the fluorescence labelled anti-CD31 antibody described herein.

Accordingly, the present invention also relates to a method for determining as to whether a compound may have an anti-angiogenic effect comprising providing a rodent, preferably a mouse, having a solid tumor, preferably a solid tumor from a human, administering to the animal an agent having a potential anti-angiogenic effect, and monitoring a potential anti-angiogenic effect in the animal by detecting a fluorescence labelled anti-CD31 antibody.

Namely, the effects of an anti-angiogenic agent can be revealed and compared by the increase or decrease of the fluorescence labelled anti-CD31 antibody signal over time. In particular, an increase or decrease of blood vessels supplying the tumor with blood can be visualized by the binding (or non-binding) of the labelled anti-CD31 antibody to CD31, i.e., the less blood vessels, the less is the signal provided by the antibody or, likewise, the more blood vessels, the more is the signal provided by the antibody. Accordingly, the less the signal, the higher is the anti-angiogenic effect of the agent.

In a third aspect, the present invention relates to a fluorescence labelled anti-CD31 antibody for use in the methods described herein.

In a fourth aspect the present invention relates to a kit for use in the methods described herein which comprises a fluorescence labelled anti-CD31 antibody and means for near-infrared fluorescence imaging to detect to the antibody in a subject. These means are described herein, for example, the MAESTRO system which is a preferred embodiment.

In a another aspect the present invention relates to the use of a fluorescence labelled anti-CD31 antibody for the preparation of a diagnostic composition for in vivo imaging tumor vasculature in a subject.

In a further aspect the present invention relates to the use of a fluorescence labelled anti-CD31 antibody for the preparation of a diagnostic composition for in vivo monitoring the therapeutic efficacy of an anti-angiogenic agent in a subject.

The embodiments provided herein are equally envisaged for the afore-described aspects, mutatis mutandis.

The Figures show:

FIG. 1: Processes in angiogenesis

(a) Angiogenesis growth factors bind to their receptors on endothelial cells and activate signal transduction pathways. (b) Matrix metalloproteinase's (MMP's) are activated and degrade the extracellular matrix enabling endothelial cells to migrate out of the pre-existing capillary wall and to proliferate. (c) Integrin αᵥβ₃ is expressed by endothelial cells facilitating their adhesion to the extracellular matrix and their migration. (d) Angiopoietin-1 (Ang1) binds to Tie2 receptor on endothelial cells. It is thought that this interaction stimulates vessel sprouting, pericyte recruitment and vessel survival and stabilization. (e) Endothelial cells release PDGF-BB, which is a chemoattractant for pericyte precursors. These cells become associated with endothelial cells and differentiate into pericytes.

FIG. 2: Balance Hypothesis for the angiogenic switch

The normally quiescent vasculature can be activated to sprout new capillaries, a morphogenic process controlled by an angiogenic switch mechanism. Changes in the relative balance of inducers and inhibitors of angiogenesis can activate the switch.

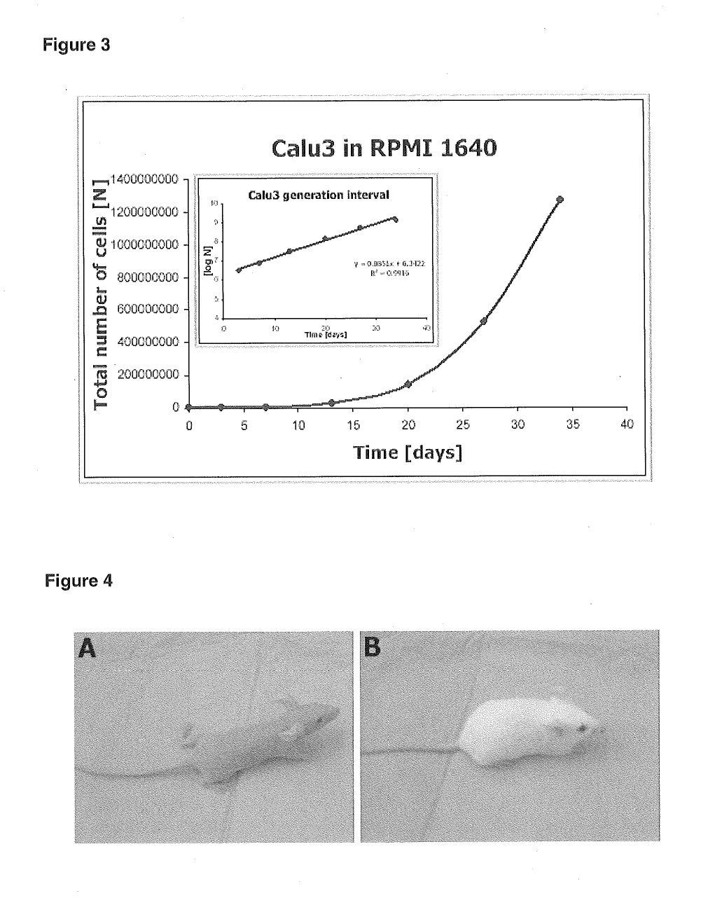

FIG. 3: Calu3 cells grown in RPM' 1640 media

At day 34 the cell population reached a total number of ˜1.3°109 which is sufficient for the inoculation of 150 mice. By the time of inoculation (cell passage number 6) the cell viability was about 84% and the logarithmic diagram reveals a generation interval of approximately 3.5 days (84 hours).

FIG. 4: Mouse models

(A) Balbc/nude mouse. (B) Scid Beige mouse.

FIG. 5: The MAESTRO-system is basically composed of three main modules that are connected to each other

(I) the Illumination Module that prepares the excitation light of a desired wavelength range, (II) the Imaging Module, where the test animal is placed and anesthetized. The emitted light of the injected probe can be detected by a CCD-camera, (III) the Working station and Analysis Module that acquires and analysis digital multispectral data.

FIG. 6: In vivo imaging and spectral unmixing with the MAESTRO system

(A) Photograph of the study animal in white light. (B) Generated RGB-picture using the red filter set. (C) Separated gray scaled image of the target specific labelled antibody (unmixed image). (D) Separated gray scaled image of the tissue autofluorescence (unmixed image). (E) Overlay of the separated grey scale images (unmixed composed image), white demonstrates tissue autofluorescence, red represents labelled antibody. (F) Illustration of signal intensity of the labelled antibody in false colors. (G) Definition of a detection limit to reduce unspecific label signals.

FIG. 7: Quantification of target specific signal intensities

Grey scaled image with an overlay showing the region that is measured. The dashed lines drawn on top of the region show the regions major and minor axis drawn through the center of gravity. The system calculates each regions area in pixel and in mm².

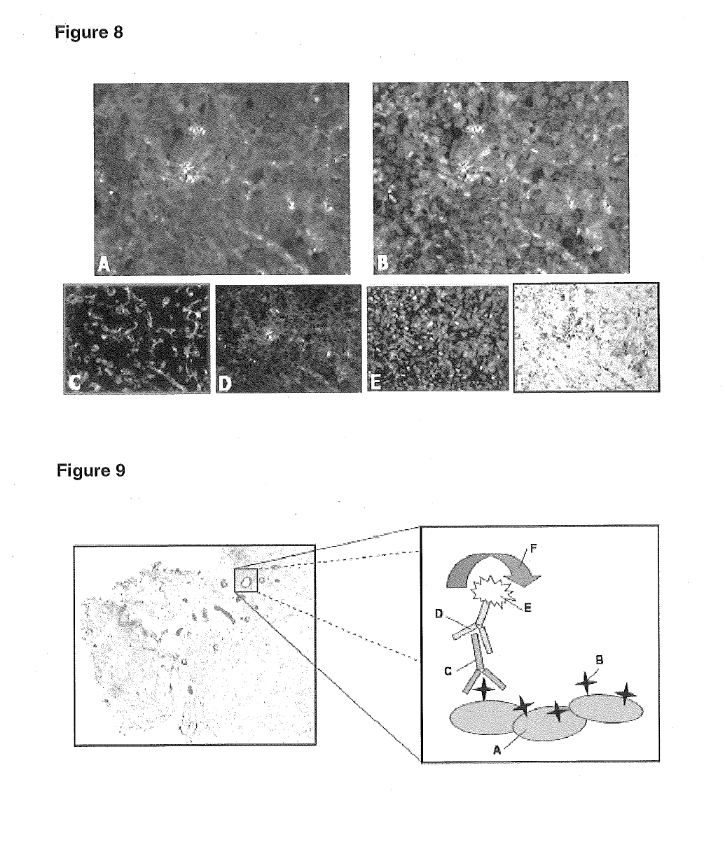

FIG. 8: Multispectral imaging with the Nuance camera

The tissue section is stained with two specific labels (Dapi, alexa647) and examined with the Nuance system. (A) RGB figure showing a restricted specific visualization because of unfiltered autofluorescence. (B) Overlay of the three specific emission spectra (composed image) after separation and visualization in high contrast colors shows an excellent visualization of each single component. (C) Signal for alexa647. (D) Erythrocyte specific autofluorescence. (E) Nuclei stained with Dapi. (F) Tissue autofluorescence.

FIG. 9: Immunhistochemistry

Detection of mouse endothelial cells using an alkaline phosphatase system. (A) Mouse endothelial cells. (B) CD31 receptor. (C) Anti-CD31 antibody. (D) Goat anti-hamster IgG secondary antibody conjugated with an alkaline phosphatase (E). (F) Visualization by incubation with permanent red solution.

FIG. 10: ex vivo quantification of blood vessel density

Pictures are taken after tumor explanation, ixation and paraffin penetration using the Nuance system (magnification 40×). (A) Original fluorescence picture. Anti-CD31-alexa610 signal is illustrated in yellow, whereas the autofluorescent erythrocytes are represented in bright blue. (B) Separated anti-CD31-alexa610 signal (yellow). (C) Transformed and reversed binary picture of anti-CD31-alexa610 (black). (D) Manually defined blood vessel regions (red).

FIG. 11: Tumor size measurement of Calu3 tumors implanted subcutaneously in nude mice

Tumors showed an exponential growth. At day 35, the tumor sizes ranged from approx. 40-200 mm3. In the randomization process, mice were optimal distributed in 6 groups with 10 mice each and they were treated with therapeutic antibodies.

FIG. 12: Photographic demonstration of tumor development and tumor activated capillary growth

At day 21 post tumor inoculation, the small blood channels (blue arrows) have extended into the pre-existing large mouse vessels (green arrows) and the tumors established a blood system to supply themselves with oxygen and crucial nutrients.

FIG. 13: Non targeted blood vessel visualization in vivo

At day 21, vascular structures around the tumors (blue arrows) were visible after tail vein injection of Qtracker705 (2 nmol/mouse) non-targeted quantum dots and visualization after 24 hr using the MAESTRO system. Quantum dots 705 are small Nan crystals exhibiting intense fluorescence with a red-shifted emission for increased tissue penetration and they are circulating up to 3 hours in the animal blood system. Tumors are characterized as black areas suggesting an establishment of a functional tumor blood system.

FIG. 14: Blood vessel distribution in the whole tumor sections

Mice were injected with unlabelled antimouse CD31 antibody 24 hr prior tumor explantation. (A) CD31 visualization (pink) at day 7 and counterstain with hematoxylin (violet). The enlarged image shows murine stroma cells (violet) migrating into the unstructured tumor tissue (bright violet). (B) 21 days post inoculation, visualization of CD31+ endothelial cells (pink) and counterstain with hematoxylin (violet) exhibited major vessels at the tumor outlines.

FIG. 15: Expression of various angiogenic growth factors during tumor development mRNA expression was determined by using 3 tumors explanted at 5 different time points. Gel electrophoresis was carried out using a 1% gel for VEGF (human) and β-actin and 1.5% for VEGF (mouse) and Ang2 (human). VEGF (human) and VEGF (mouse) revealed a tumor size dependent expression pattern whereas VEGF (mouse) levels stayed the same.

FIG. 16: Injectable imaging agents for monitoring angiogenesis

Animals were injected with 50 μg/mouse of mab3-alexa647, anti-CD31-alexa610 and mab7-alexa700 and 2 nmol/mouse of Integrisense680 and imaged 24 hr post-agent-injection. Mice A and D are Scid beige mice bearing a Kp14 tumor whereas B and C represent Balbc/nude mice bearing a Calu3 tumor. Tumors were explanted, fixed in formalin for 24 hr and embedded in paraffin. Nuance images were taken with a 40× magnification. (A-D) In vivo NIRF-imaging. Deep red represents regions of high reagent concentrations whereas blue represents regions of low concentration. (A-40×-D-40×) Nuance ex vivo imaging allowed to localize the imaging reagents (red fluorescence) in the tumors. Bright blue fluorescence (DAPI) shows cell nuclei. Yellow (B-40×) and blue (C-40×) represent tumor tissue.

FIG. 17: In vivo live imaging with two target specific antibodies

(A-C) Separation of CD31-alexa610 (green fluorescence) and Herceptin-alexa700 (red fluorescence) in two single images. (D-40×) Ex vivo Nuance image revealed Herceptin (red fluorescence) targeting tumor cells and blood vessels (green fluorescence) are mainly observed at the tumor edges. Yellow fluorescence represents erythrocytes. (E-F) The intensity of anti-CD31-alexa610 is much weaker than the one originating from Herceptin-alexa700. Anti-CD31-alexa610 is well distributed all over the mouse body but mainly in the tumor, lung and bladder. Herceptin-alexa700 is less distributed and highly located in the tumor and the liver/kidneys.

FIG. 18: Time-dependent antibody distribution in human xenograft

Scid beige mice bearing H322M tumors were injected with IntegriSense680 (2 nmol/mouse) and imaged at different time points. The kinetic revealed that signals from circulating reagents and unspecific bindings decrease progressively over time.

FIG. 19: Reagent signal intensities dependent upon their tissue depth

50 μg/mouse of mab3-alexa647 were i.v. injected in Calu3 bearing Balbc/nude mice. NIRF imaging revealed highest concentration within the tumor region. Nuance ex vivo imaging demonstrated similar signal intensities in (A) liver, (B) kidney and (C) spleen. (D) Agent concentration in lung was lower than in tumor and excretory organs.

FIG. 20: Growth curve of treatment groups 1-6

All groups revealed a tumor inhibition up to 50% with mab 4 relative to the control mab.

FIG. 21: Body weight curves

FIG. 22: Statistical analysis of therapy

The analysis was carried out relative to the control mab by using the AUC evaluation of the SAS program.

FIG. 23: Comparison of tumor vasculature of different treatment groups

Mice were injected with anti-CD31 antibody (50 μg/mouse), imaged 24 hrs post-injection and compared with the compare imaging tool.

FIG. 24: Relative change of CD31 signal during therapy

Treatment groups exhibited an increase of labelled anti-CD31 signals within the tumor regions in the first two weeks of treatment (yellow bars). In contrast, between day 79 and 47, treatment with mab 2+3 and mab 4 reduced tumor vasculature significantly, while control group and treatment with mab 2 accelerated tumor blood vessel formations (violet bars).

FIG. 25: Fluorescence microscopy for anti-CD31 and histochemical staining

After treatment for 44 days, mice were injected with anti-CD31 antibodies, sacrificed and tumors were removed. Lower row, represents the ex vivo fluorescence studies. Blood vessels stained with anti-CD31 antibody (yellow) were much more present in the group treated with the control mab. Another evidence for tumor capillaries is the appearance of autofluorescent erythrocytes (bright blue). Upper row shows HE-stained tumor sections. Control tumors revealed some necrotic regions, whereas tumor necrosis was extremely accelerated in those groups treated with anti-angiogenic antibodies.

FIG. 26: Quantification of blood vessel density ex vivo.

FIG. 27: mRNA expression pattern of various angiogenic growth factors during therapy mRNA expression was determined by using 3 tumors at day 79. Gel electrophoresis was carried out using a 1% gel for VEGF (human) and β-actin and 1.5% for VEGF (mouse) and Ang2 (human).

FIG. 28: Anti-mouse-CD31 ex vivo fluorescence imaging

Tumors are explanted 24 hrs post-agent injection. Anti-mouse-CD31 antibody (red fluorescence) is the best imaging agent for monitoring tumor angiogenesis. It reveals precise contours of tumor blood vessels. Blue fluorescence represents tumor cell nuclei and yellow fluorescence corresponds to erythrocyte autofluorescence.

FIG. 29: FMT three-dimensional imaging technology

(A) Two-dimensional imaging shows highest antibody concentrations in the tumor region (whit circle). (B) FMT image front side. (C) FMT image +30° side. (D) FMT image −30° side.

FIG. 30: Tumor size measurement and ex vivo analysis for control mab, mab 2 (Treatment Group 3) and mab 4 (Treatment Group 6).

FIG. 31: Detection of blood vessels by use of Cy₅ labeled anti-CD31

(A) Anti-murine CD31 antibody labeled with Cy₅ was injected i.v. at a concentration of 50 μg/mouse (2 mg/kg) in Calu3 xenograft BALB/c nude mice. After 48 hrs in vivo image was acquired.

(B) 15 min thereafter tumor was explanted and tissue slide section was analyzed by fluorescence microscopy.

Erythrocytes (in yellow) within a vessel is an indication that vessels are functional Vessel wall (in red) stained with Cy5 labeled anti-CD31 antibody

The following Examples should not be construed as a limitation of the present invention, but merely illustrate the invention.

### EXAMPLES

### 1. Cell Lines

### 1.1 Calu3 (Caucasian, Adenocarcinoma, Lung, Human)

The human NSCLC (Non-small cell lung carcinoma) cell line Calu3 was obtained from Chugai (Kamakura, Japan). Its characteristics are the slow growing pattern in the animal studies as well as in cell culture, where the cells grow in a monolayer and are strongly attached to the bottom of the cell culture flasks. In vivo, the tumors exhibit a high vascularization pattern and thus, it was the major cell type used in this work. The cell line was cultured, injected into mice, imaged with different angiogenic probes and treated with therapeutic antibodies developed by Roche.

### 1.2 KPL-4

The breast cancer cell line, KPL-4, was isolated from the malignant pleural effusion of a breast cancer patient with an inflammatory skin metastasis. The cell line was also obtained from Chugai (Kamakura, Japan). KPL-4 xenografts were used for the optimization of the in vivo imaging parameters reagents specific for monitoring angiogenesis.

### 1.3 H322M

The second “Non-small cell lung cell line” was H322M. It also originates from Chugai (Kamakura, Japan). This cell line was applied in the same way like the KPL-4 xenograft used for the optimization of the in vivo life imaging parameters.

### 2. Antibodies and Fluorescence Imaging Agents

TABLE 2
Overview of antibodies and fluorescence imaging agents

| | Concentration | | Manufacturer |
|---|---|---|---|
| Fluorescence labelled | | | |
| Antibodies | | | |
| Mab7-alexa700 | 50 | μg/mouse | Roche Diagnostics |
| | | | GmbH, Mannheim |
| Mab3-alexa647 | 50 | μg/mouse | Roche Diagnostics |
| | | | GmbH, Mannheim |
| Anti-mouse-CD31-alexa610 | 50 | μg/mouse | Acris Antibodies, |
| | | | Herfold |
| Non-peptide Agents | | | |
| IntegriSense ™ 680 | 2 | nmol/mouse | VisEn Medical, |
| | | | Bedford |
| Qtracker 705 Nontargeted | 600 | nmol/mouse | Invitrogen, Carlsbad |
| Quantum Dots | | | |
| Antibodies for | | | |
| Immunohistochemistry | | | |
| anti-mouse-CD31 (Host: | 50 | μg/mouse | Acris Antibodies, |
| Armenian hamster) | | | Herfold |
| Goat anti-armenian hamster | 1/200 | | Dianova, Hamburg |
| IgG ALP-conjugated | | | |
| Therapeutic Antibodies | | | |
| Control mab (anti-IgE | 10 | mg/kg | Roche Diagnostics |
| antibody) | | | GmbH, Mannheim |
| Mab 1 | 5 | mg/kg | Roche Diagnostics |
| | | | GmbH, Mannheim |
| Mab 2 | 10 | mg/kg | Roche Diagnostics |
| | | | GmbH, Mannheim |
| Mab 3 | 10 | mg/kg | Roche Diagnostics |
| | | | GmbH, Mannheim |
| Mab 4 | 13.3 | mg/kg | Roche Diagnostics |
| | | | GmbH, Mannheim |

### 3. PCR Primers

For mRNA expression studies, RT-PCR was carried out using target specific primers obtained from TIB MOLBIOL GmbH (Berlin). Human VEGF-A gene expression reveals 7 different splice variants and 6 of them are detected by using the VEGF-A specific primers.

TABLE 3
Overview of primer sequences and annealing
temperatures

| | Anneal- | |
|---|---|---|
| | ing | |
| | temp. | |
| Primer | [° C.] | Sequence |
| (human specific) | | |
| VEGF-A | 55 | FORWARD: |
| | | 5′-GCACCCATGGCAGAAGGAGG |
| | | A-3′ |
| | | (SEQ ID NO: 1) |
| | | REVERSE: |
| | | 5′-TCACCGCCTCGGCTTGTCAC-3′ |
| | | (SEQ ID NO: 2) |
| Angiopoietin 2 | 50 | FORWARD: |
| | | 5′-CAGATTGTTTTCTTTACTTC-3′ |
| | | (SEQ ID NO: 3) |
| | | REVERSE: |
| | | 5′-CTGATATTGCTTCTTTCCTA-3′ |
| | | (SEQ ID NO: 4) |
| Beta-actin | 55 | FORWARD: |
| (control) | | 5′-ATGGATGATGATATCGCCGC |
| | | G-3′ |
| | | (SEQ ID NO: 5) |
| | | REVERSE: |
| | | 5′-CTAGAAGCATTTGCGGTGGACGA |
| | | TGGAGGGGCC-3′ |
| | | (SEQ ID NO: 6) |
| (mouse specific) | | |
| VEGF-A | 63 | FORWARD: |
| | | 5′-TACTGCTGTACCTCCACCTCCAC |
| | | CATG-3′ |
| | | (SEQ ID NO: 7) |
| | | REVERSE: |
| | | 5′-TCACTTCATGGGACTTCTGCTC |
| | | T-3′ |
| | | (SEQ ID NO: 8) |
| Angiopoietin 2 | 50 | FORWARD: |
| | | 5′-GGTTAGAGATAGTGTCGACA |
| | | G-3′ |
| | | (SEQ ID NO: 9) |
| | | REVERSE: |
| | | 5′-GACTGTAGCTGCTCGCTGTGCT |
| | | TG-3′ |
| | | (SEQ ID NO: 10) |

By using these primers, the desired fragments should have a size of:

TABLE 4
PCR-fragment sizes

| | Splice variants | Size of fragment(s) [bp] |
|---|---|---|
| Primer (human specific) | | |
| VEGF-A | 6 | 620, 569, 551, 497, 462, 365 |
| Angiopoietin 2 | 1 | 100 |
| Beta-actin (control) | 1 | 1128 |
| Primer (mouse specific) | | |
| VEGF-A | 1 | 78 |
| Angiopoietin 2 | 1 | 264 |

### 4. METHODS

### 4.1 Cell Culture

Calu3 cells are cultured in T75 cell culture flasks with RPM! 1640 media supplemented with 10% fetal bovine serum (FBS) and 2 mM L-Glutamine in 5% CO₂ at 37° C. The slow-growing NSCLC cells are split in 7 day periods by using trypsin, morphological assessment was performed using a normal light microscope and counted by Vi-CelI™ Cell Viability Analyzer. The cells reveal an exponential growing pattern with a generation interval of approx. 3.5 days and at day 35, cells are trypsinized, washed, counted and diluted to 5*106/100 μl.

### 4.2 Mouse Models

To avoid an immune reaction against the injected human tumor cells, female Balbc/nude mice (see FIG. 6 A) are selected, 4-5 weeks of age, from Bomholtgard (Ry, Denmark). These animals lack a thymus, are unable to produce mature T-cells, and are therefore immunodeficient and appropriate for xenograft studies. For preliminary tests, Scid Beige mice (see FIG. 6 B), from Charles River (Sulzfeld, Germany), injected with KPL4 or H322M tumors are used. Scid Beige mice possess both of the genetic autosomal recessive mutations “Scid” and “Beige”. “Scid” mice show severe combined immunodeficiency affecting both B and T lymphocytes whereas the “Beige” mutation affects only the NK-cells. The Scid Beige mice are shaved for imaging experiments in order to reduce autofluorescence signals resulting from the body fur. All animals are provided free access to food and water throughout the whole time of study. For in vivo live imaging experiments, anesthesia is carried out through inhalation of Isofluran inserted in an appropriate anesthesia box. All studies are performed according to animal welfare regulations and have been approved by local authorities.

### 4.3 Tumor and Transponder Inoculation

The exponentially growing Calu3 cells are harvested, washed in PBS and injected subcutaneously into the right flank region of the anesthetized mice by an experienced lab technician, in a volume of 0.1 ml and a total number of 5*106 tumor cells. For easy identification of the Balbc/nude mice during the whole study, they are injected with a transponder that is also injected subcutaneously using a transponder injection device. All experimental studies were carried out according to committed guidelines (GV-Solas, Felasa, TierschG). Experimental study protocols were reviewed and approved by local government (registration no. 211-2531.2-22/2003).

### 4.4 Tumor Size and Body Weight

Tumor size and body weight are measured in mm and g by using a caliper and a balance. Results are transferred to a local data base. The formula for tumor volume calculation used in the program is an approximation for the calculation of ellipticalshaped tumors. It comprises the length L (longest dimension) and width W (shorter dimension, parallel to the mouse body) of the tumors.

V=0.5×L×(W²)

### 4.5 Randomization

To obtain statistically well distributed groups for the treatment with therapeutic antibodies, the animals need to be randomized. This random and homogenous distribution is carried out using the internal software based system called “Phasis” (Pharmacology Animal study Information System). Shortly before the start of therapy, mice are grouped depending on their body weight and their size of the tumor. The system finds the smallest distance between largest and smallest tumors in one group. The randomization tool gives a homogenous and, most important, a comparable grouping as a start point of therapy.

### 4.6 Parenteral Injection of Antibodies

Intraperitoneal (i.p.) injection of therapeutic antibodies during therapy is carried out using a 1.0 ml syringe and an 18 G-canula and applied in the lower part of the abdominal cavity of the mice in a volume of 200 μl. Intravenous (i.v.) application of fluorescent labelled antibodies is performed using a 1.0 ml syringe and a 27 G-canula and injected into the mouse tail vein in a volume of 200 μl.

4.7 NIRF In Vivo Live Imaging with MAESTRO®

The MAESTRO system is a planar fluorescence-reflecting-imaging system that allows a noninvasive in vivo fluorescence measurement. In this multispectral analysis, a series of images are captured, at specific wavelengths. The range of wavelengths captured should cover the expected spectral emission range of the label present in the specimen. The result will be a series of images called “image cube” and it is the data within this series of images that is used to define the individual spectra of both autofluorescence and specific labels. Many labels of biological interest have emission spectra that are so similar that separation using expensive narrow band filters is difficult or impossible. A single long pass emission filter replaces a large collection of emission filters. In addition to the natural autofluorescence of the skin, fur, sebaceous glands, there is also distinct autofluorescence from comensal organisms (fungi, mites, etc.) and ingested food (chlorophyll). Multispectral analysis is able to separate all of these signals from the specific label applied to the specimen through the mathematically disentanglement of the linear signal mixture (unmixing) of the emitted fluorescent lights as long as the emission spectrum of the desired signal and of the autofluorescence are known.

### Measurement

The illumination module is equipped with a xenon lamp (Cermax) that excites white light. Through a downstream connected excitation filter (chosen by the experimenter), the light is delimitated to a, for the experiment, desired wavelength range and conducted via an optical fiber into the imaging module. In here, the restricted light is partitioned into four optical fibers that illuminate the anesthetized test animal. The MAESTRO system chooses the optimal exposure time automatically, so that there is no risk of overexposure. The emitted fluorescence light of the activated fluorescent probe is selected with an emission filter (see Table 1) and conducted through a liquid crystal (LC) to a high sensitive, cooled CCD-camera. The liquid crystal enables the camera a selective picture recording of a specific wavelength. The wavelength measurement range depends on the selected filter set (blue, green, yellow, red, deep red, NIR) and pictures are recorded in steps of 10 nm. The spectral information of each single picture is combined in one “picture package” that is called “image cube” (see Table 1).

### Analysis

Each recording compose of 12 bit black-and-white pictures that can be illustrated in 4096 different gray scales and therefore it is possible to discriminate between smallest differences in emission intensities. In contrast, the human eye is able to distinguish between 30-35 grey scales. Those values for the emission intensities (grey scales) are plotted against the wavelength range and as a result, we obtain the emission spectra of each probe and the tissue autofluorescence. The software subdivides the three fundamental colors (red, green, blue) to the wavelength range used for the imaging cube whereby the black-and-white pictures turn into colored image (RGB image, see FIG. 8F). Out of these acquired multispectral information the system is able to differentiate between injected probes and autofluorescence of any source. The program is using a spectral library, where the single spectra of each pure probe and the spectra acquired by imaging the study animals (Balbc/nude or Scid Beige mice) without any injection (mouse autofluorescence). By knowing the exact spectra of the pure imaging and of the autofluorescence, the system is able to filter the whole image for the desired spectra and assign a color to each of them. The originated image (unmixed composite image) shows the present spectra in different colors. To visualize the intensity distribution of the probe signal, it is possible to illustrate the signal in false colors (see FIG. 6F), whereas low intensities are blue and regions of high intensities are red. Besides that, one can define a detection limit for the signal intensity of the probe, which allows reducing the signal of circulating probes and unspecific bindings (see FIG. 6G).

### Comparison and Quantification

The MAESTRO's ability to compare fluorophore regions of an image makes it easy to compare the tumor fluorescent signal intensities during therapy. The program provides tools for the comparison of different signal intensities in tumor regions (compared images). Since all images are taken at optimal exposure times, they differ depending on the strength of signals. For a reliable comparison, the pictures are standardized to one exposure time, resulting in an illustration of differences in signal intensities. By manually drawing and modifying measurement regions, signal intensities can be quantified in intensity values. Once a measurement area is selected around the tumor, it can be cloned and moved to the next image to be compared with. Each region is calculated in pixels and mm2 based on the current settings (stage height and binning). As a result, it gives information about the average signal, total signal, max. signal and average signal/exposure time (1/ms) within the created measurement area.

### 4.8 Tumor Explantation

Animals are sacrificed and tumors are explanted according to committed guidelines (GVSolas, Felasa, TierschG). After sacrificing the animals, tumors are explanted using a scalpel and transferred into an embedding cassette or flash frozen in liquid nitrogen with isopentan for immunohistochemical applications and without isopentan for PCR applications.

### 4.9 Tumor Fixation and Paraffin Sections

Explanted tumors are enclosed in an embedding cassette and are incubated under continuous agitation in formalin for approximately 24 hours. Thereafter, formalin is discarded and tumors washed with dest. water followed by dehydration of the tumors and penetration of paraffin (see Table 5). All the incubation steps are carried out using the Tissue Tek® VIP Vacuum Infiltration Processor.

TABLE 5
Dehydration and paraffin coating

| Material | Incubation time [hr] | Temperature [° C.] |
|---|---|---|
| 3 × Ethanol 70% | 1.5 | Room temp. |
| 2 × Ethanol 95% | 1.5 | Room temp. |
| 2 × Ethanol 100% | 1.5 | Room temp. |
| 2 × Xylol 100% | 1.5 | Room temp. |
| 4 × Paraffin | 1 | 60 |

After paraffin penetration, tumors are embedded in liquid paraffin with the Tissue Tek® Paraffin embedding station to a final histological block. Paraffin sections in a range of 2-8 μm of thickness are obtained from these blocks using a microtome. They are cut, uptaken on glass slides, air dried over night at 37° C. followed by de-paraffinization and HE-staining (see table 10). Slides are examined with the Nuance system.

### 4.10 Hematoxylin-Eosin Staining

Hematoxylin and eosin stain are of the most commonly used stains in histology to examine abnormal tissue variations in biopsies or surgery compounds. Hematoxylin is extracted from the wood of the logwood tree. When oxidized it forms haematein, a compound with rich blue-purple color, and is used, together with a suitable mordant (most commonly Fe(III) or AI(III) alums) forming hemalum (see Table 6), to stain cell nuclei prior to examination under a microscope. During staining, the positively charged alum complexes interact with the negatively charged phosphate groups of nucleic acids. The final blue color is obtained by blueing with tap water. The staining should be examined for desired staining grade using the light microscope and stopped at favored staining intensity.

TABLE 6
Hemalum solution

| Material | Amount [g] |
|---|---|
| Citric acid | 1 |
| Hematoxylin | 1 |
| Sodium iodide | 0.2 |
| Aluminium | 50 |
| potassium sulfate | |
| Chloral hydrate | 50 |

Eosin is a synthetic red dye often used as the counterstain to hematoxylin staining. Eosin is an acidic dye and negatively charged, so that it is bound through electrostatic adsorption to positive groups and shows up in the basic parts of the cells (collagen, basic parts of the cytoplasm).

TABLE 7
De-paraffinization and HE-staining

| | Incubation time |
|---|---|
| Material | [min] |
| Xylol 100% | 5 |
| Xylol 100% | 5 |
| Xylol 100% | 5 |
| Ethanol 100% | 2 |
| Ethanol 90% | 2 |
| Ethanol 80% | 2 |
| Ethanol 70% | 2 |
| dest. water | 5 |
| hemalum | 1 |
| tap water | 1-20 |
| dest. water | rinse |
| Eosin (1%) | 1 |

### 4.11 Supplying of Sections

After HE-staining, the tissue sections are dehydrated (see Table 8), supplied with approximately 100 μl Cytoseal™ mounting media and covered with glass cover slips.

TABLE 8
Dehydration and supplying of cover slips

| | Incubation time |
|---|---|
| Material | [min] |
| Ethanol 70% | 5 |
| Ethanol 80% | 5 |
| Ethanol 90% | 5 |
| Ethanol 100% | 2 |
| Xylol 100% | 2 |

### 4.12 Nuance® Ex Vivo Imaging

The multispectral imaging technique with the Nuance camera acquires high sensitive images on paraffin or cryosections and allows quantitative and sensitive measurements of subtle differences in spectral emissions. Nuance has two main advantages. First, it is able to do brightfield imaging and it can also act as an excellent colour camera. Secondly, in fluorescence microscopy, in samples which are autofluorescent, like most tissue sections, Nuance is separating tissue auto-fluorescence from signals of interest. The function is similar to the MAESTRO system using a filter set and multispectral analysis.

### Measurement

The paraffin slides are placed at the specimen stage and brought into focus. The UV-lamp of the fluorescence microscope emits a broad spectrum of UV-light. The excitation filter delimitates the light to the desired wavelengths range and it is focused through a beam splitter on the tissue sections. The fluorescent probes, contained in the sections, absorb light and emit light of fluorophore specific wavelengths. The optimal exposure time is set up by the Nuance system automatically. The downstream connected emission filter allows just defined wavelengths to pass and they are recognized by the camera. The Nuance camera is a high sensitive, cooled CCD-camera containing a liquid crystal filter. This camera system is identical to the described Maestro system (Chapter 4.7). Multispectral imaging and analysis are based on the same background allowing to separate fluorescent probes with similar emission spectra and the removal of tissue autofluorescence.

### Analysis

The Nuance system is also working with a spectral library. Pure single spectra of the fluorescent probes and of the autofluorescence are needed to separate and visualize the different signals (see FIG. 8). For doing this, the single spectra of each fluorophore are measured using the appropriate filter set as well a tissue section without any labeling to reveal the tissue autofluorescence. These spectra are transferred into the system, separated and visualized in different colors (see FIG. 8).

### 4.13 Immunohistochemistry

To visualize tumor blood vessels through the whole tumor section, an unlabelled endothelial antibody (hamster anti-mouse-CD31) is injected into the mice in a concentration of 50 μg/mouse. Tumors are explanted at 24 hours post-injection and sectioned after flash freezing in liquid nitrogen and isopentan. The unlabelled vessel marker is identified using a secondary antibody (goat anti-hamster IgG) alkaline phosphatase system. A slide scanner allows the visualization of the whole tumor section.

### Cryosections

For Immunohistochemistry applications, tumors are flash frozen immediately after excision in Isopentan and liquid nitrogen and held at −20° C. until sectioning. Briefly, 10 μm tumor cryosections are cut at the optimum cutting temperature of −18° C. with the Cryostat (2800 Frigocut), fixed with acetone for one minute, air dried and used for Immunohistochemistry application.

### Immunostaining Procedure and Slide Scan

Sections are adjusted in Dako wash buffer, for 5 minutes and blocked with 10% goatserum for 20 minutes to prevent unspecific bindings of the secondary antibody. The slides are rinsed twice for 2 minutes prior to the incubation of the goat anti-hamster IgG secondary antibody (AP conjugated) for 1 hour and a 1/200 dilution. The sections are washed three times for 5 minutes with Dako wash buffer containing 0.025% Tween. For the visualization, the slides are incubation twice for 10 minutes with the permanent red solution, rinsed in dest. water and blued for at least 5 minutes. Tissue sections are dehydrated (see Table 8), supplied with approximately 100 μl Cytoseal™ mounting media and covered with glass cover slips. The MIRAX SCAN is equipped with an external scan camera and allows digitizing the slides. It detects the sample on the slide and scans it in constant high quality and offers the possibility to quantify the whole tumor tissue sample.

4.14 mRNA Expression Profiling

RT-PCR is carried out to determine the expression profile of various angiogenic growth factors (VEGFA, ANG2) during tumor development and tumor blood vessel growth. Mouse and human specific primers are used to reveal the balance between the RNA expression resulting from human material and the expression resulting from mouse itself. RNA extraction The explanted tumors are placed in a MagNA Lyser LC Green Bead and snap frozen in liquid nitrogen. By adding the appropriate volume of lysis buffer (depending on the weight of the tumor), the sample is homogenized in the fast prep instrument until complete disruption of the tumor tissue. The centrifugation step is pelleting the debris in the bottom of the tubes making it easy to take the supernatant and place it into the sample cartridge to start the mRNA extraction with the MagNA pure LC system. The system is working automatically by choosing the program for the isolation of mRNA from tissue samples.

MagNA Pure LC System Procedure Using the Magna Pure Lc mRNA Isolation Kit II (Tissue)
1) Lysate supernatants are pipetted into the sample cartridge
2) Samples are diluted with the Capture buffer containing biotin-labelled oligo(dT), which is hybridizing to the poly(A) tale of mRNA's
3) After addition of SMPs (Streptavidin Magnetic Particles) the oligo(dT)-mRNA complex is captured and immobilized onto the bead surface
4) Genomic DNA is removed by incubation with DNase I
5) SMPs with the adsorbed mRNA are recovered from the wells of the processing cartridge by applying a magnet from the outside of reaction tubes
6+7) Repeatedly washing steps with wash buffer removes unbound substances like proteins, cell membranes and reduces the chaotropic salt concentration
8) The purified mRNA is eluted at 70° C. from the SMPs in the wells of the elution cartridge, whereas the SMPs remain in the reaction tubes
   mRNA Quantification

Purified mRNA yields are obtained using the Nanoprop 1000 Spectrophotometer. It enables high accurate UV/Vis analysis by using just 1 μl without any dilutions. mRNA is measured at 260 nm and 280 nm and as a result, the program gives the mRNA yield in ng/μl and a value for mRNA quality (high quality: OD 260/280 ratio of 1.8-2.2).

### RT-PCR

RT-PCR is carried out in two steps: First strand cDNA synthesis using the first strand cDNA synthesis Kit followed by a polymerase chain reaction using the High Fidelity PCR Master Mix.

Components for cDNA Synthesis

mRNA is transcribed into single-stranded cDNA at the 3″-end of the poly(A)-mRNA by using the Oligo-p(dT) 15 primer.

TABLE 9
Components for first strand cDNA synthesis

| Component | Volume/Concentration | |
|---|---|---|
| 10x Reaction Buffer | 2.0 | μl |
| 25 mM MgCl₂ | 4.0 | μl |
| Deoxynucleotide Mix | 2.0 | μl |
| Oligo-p(dT)₁₅ primer | 2.0 | μl |
| RNase Inhibitor | 1.0 | μl |
| AMV Reverse | 0.8 | μl |
| Transcriptase | variable | |
| Sterile water variable | 500 | ng |
| RNA sample | | |
| Total volume | 20 | μl |

cDNA Synthesis Reaction:

| | | |
|---|---|---|
| 10 min | 25° C. | Annealing |
| 60 min | 42° C. | Transcription |
| 5 min | 99° C. | Denature AMV Transcriptase |
| 5 min | 4° C. | |

### Components for PCR:

TABLE 10
Components for polymerase chain reaction

| Component | Volume/Concentration |
|---|---|
| PCR Master Mix 20 μl | 20 μl |
| Primer forward 1 μl | 1 μl |
| Primer reverse 1 μl | 1 μl |
| Dest. water 8 μl | 8 μl |
| cDNA template | 20 μl |
| Total volume | 50 μl |

### PCR Reaction:

| | | | |
|---|---|---|---|
| 4 min | 94° | C. | Denaturation |
| 1 min | 94° | C. | Denaturation |
| 1 min | X° | C. | Annealing 35 cycles |
| 2 min | 72° | C. | Elongation |
| 10 min | 72° | C. | Elongation |

The annealing temperature differs from primer to primer (see Chapter 3).

### Gel Electrophoresis

The separation and visualization of the fragments is carried out by electrophoretic separation in an ethidium bromide containing agarose gel (1-2%). The crosslinked polymers in the gel exhibit a porosity based on the fragments weight and composition. Electromotive force is used to move the nucleic acids through the gel matrix. By placing the samples in wells and applying an electric current, the molecules will move through the gel in different rates, determined by their mass to charge ratio, towards the cathode. Ethidium bromide, a positively charged fluorescent agent, intercalates in the base pairs of nucleic acids, during the run, to visualize the separated fragments. The gel is exposed to UV-light and a camera records the fluorescent image as a photograph. A molecular size marker is used to identify the approximate size of the separated fragments.

### 4.14 Statistical Analysis

Data regarding primary tumor growth were statistically analyzed. Briefly, in a randomized two-sample design the treatment-to-control ratio (TCR):

$$TCR = \frac{{\overline{V}}_{treated}}{{V}_{control}} \_$$

and its two-sided non-parametric (1-á) confidence interval (CI) was determined. The calculations were performed with the special SAS program TUMGRO (version 3.0) using SAS version 9.1 (SAS Inc. Cary,). The system allows an “endpoint” evaluation (EP) and an “area under the curve” evaluation (AUC) of the measured data. EP evaluation is using the values of the last day of tumor measurement, whereas AUC evaluation includes all data.

### 4.16 Ex Vivo Blood Vessel Quantification

The quantification of blood vessel density in paraffin sections is carried out using a custom written MATLAB script. The basic idea behind the program is to separate the anti-CD31 signal by using a k-Means-clustering (see FIG. 10B). The filtered specific signal is then transformed into a binary image and “eroded” to remove disturbing signals (erosion function). After reversing the picture (see FIG. 100), it is possible to manually define the regions of blood vessel appearance. The program marks the region of interest in red (see FIG. 10D) and calculates the percentage of blood vessel regions within the recorded picture.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

1. A non-invasive method of in vivo imaging tumor vasculature in a subject comprising detecting a fluorescence labelled anti-CD31 antibody.

2. A non-invasive method of in vivo monitoring the therapeutic efficacy of an anti-angiogenic agent in a subject comprising detecting a fluorescence labelled anti-CD31 antibody.

3. The method of any of the preceding claims, wherein the subject is to be administered the fluorescence labelled anti-CD31 antibody prior to detecting said antibody

4. The method of any of the preceding claims, wherein the subject suffers from cancer.

5. The method of any of the preceding claims, wherein the subject has a solid tumor.

6. The method of claim 5 wherein the solid tumor of the subject is a xenograft, preferably a human xenograft.

7. The method of any of the preceding claims, wherein the subject is to be treated with an anti-angiogenic agent.

8. The method of any of the preceding claims, wherein detecting is accomplished by near-infrared fluorescence imaging (NIRF).

9. The method of claim 8, wherein near-infrared fluorescence imaging includes fluorescence reflectance imaging (FRI) or fluorescence-mediated tomography (FMT).

10. The method of claim 7, wherein the anti-angiogenic agent is an inhibitor of VEGF-A, VEGF-B, VEGF-C, VEGF-D, VEGFR1, VEGFR2 or Angiopoietin.

11. The method of claim 10, wherein the inhibitor is an antibody, siRNA or a small molecule.

12. A fluorescence labelled anti-CD31 antibody for use in any of the methods defined in the preceding claims.

13. A kit for use in any of the methods defined in the preceding claims comprising a fluorescence labelled anti-CD31 antibody and means for near-infrared fluorescence imaging to detect said antibody in a subject.

14. Use of a fluorescence labelled anti-CD31 antibody for the preparation of a diagnostic composition for in vivo imaging tumor vasculature in a subject.

15. Use of a fluorescence labelled anti-CD31 antibody for the preparation of a diagnostic composition for in vivo monitoring the therapeutic efficacy of an anti-angiogenic agent in a subject.


SEQUENCE LISTING (11 sequences)

SEQ ID NO: 1 - DNA, 21 bp, artificial sequence
  primer sequence derived from human VEGF-A
gcacccatggcagaaggagga

SEQ ID NO: 2 - DNA, 20 bp, artificial sequence
  primer sequence derived from human VEGF-A
tcaccgcctcggcttgtcac

SEQ ID NO: 3 - DNA, 20 bp, artificial sequence
  primer sequence derived from human Angiopoietin 2
cagattgttttctttacttc

SEQ ID NO: 4 - DNA, 20 bp, artificial sequence
  primer sequence derived from human Angiopoietin 2
ctgatattgcttctttccta

SEQ ID NO: 5 - DNA, 21 bp, artificial sequence
  primer sequence derived from human Beta-actin
atggatgatgatatcgccgcg

SEQ ID NO: 6 - DNA, 33 bp, artificial sequence
  primer sequence derived from human Beta-actin
ctagaagcatttgcggtggacgatggaggggcc

SEQ ID NO: 7 - DNA, 27 bp, artificial sequence
  primer sequence derived from mouse VEGF-A
tactgctgtacctccacctccaccatg

SEQ ID NO: 8 - DNA, 23 bp, artificial sequence
  primer sequence derived from mouse VEGF-A
tcacttcatgggacttctgctct

SEQ ID NO: 9 - DNA, 21 bp, artificial sequence
  primer sequence derived from mouse Angiopoietin 2
ggttagagatagtgtcgacag

SEQ ID NO: 10 - DNA, 24 bp, artificial sequence
  primer sequence derived from mouse Angiopoietin 2
gactgtagctgctcgctgtgcttg

SEQ ID NO: 11 - protein, 738 aa, Homo sapiens
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQLLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT